SEQUENCE LISTING (1 sequence)

SEQ ID NO: 1 - RNA, 25 nt, synthetic construct
  source 1..25: mol_type = other RNA; note = antisense strand of siRNA
tttagtaagatccagcatcacattc

(19) United States
(12) Patent Application Publication
(10) Pub. No.: US 2026/0248824 A1
(43) Pub. Date: Aug. 27, 2026

(54) LIPID NANOPARTICLES

(71) Applicants: SHIZUOKA PREFECTURAL UNIVERSITY CORPORATION, Shizuoka (JP); TOKUSHIMA UNIVERSITY, Tokushima (JP)

(72) Inventors: Takanori KANAZAWA, Tokushima (JP); Yukiko KANEKO KUROHANE, Shizuoka (JP)

(21) Appl. No.: 19/489,656

(22) PCT Filed: May 31, 2024
(86) PCT No.: PCT/JP2024/019985

§ 371 Date: Dec. 3, 2025

(30) Foreign Application Priority Data
Jun. 8, 2023 (JP) 2023-094594

(51) Int. Cl. A61K 31/661; A61K 9/1272; A61K 9/51; A61K 47/28; A61K 47/34

(52) U.S. Cl. CPC A61K 31/661; A61K 9/1272; A61K 9/5123; A61K 47/28; A61K 47/34

(57) ABSTRACT

Provided are lipid nanoparticles showing excellent drug migration to the pancreas.

The lipid nanoparticles according to the present invention are lipid nanoparticles for delivering a drug to a target tissue, comprising: (a) a phosphatidylcholine-type phospholipid having an unsaturated fatty acid chain with 16 to 24 carbon atoms, and (b) a polyethylene glycol-modified lipid, wherein the target tissue is the pancreas.

### FIELD OF THE INVENTION

The present invention relates to lipid nanoparticles used for delivering a drug to a target tissue, and in particular relates to lipid nanoparticles used for delivering a drug to the pancreas.

### DESCRIPTION OF RELATED ART

The pancreas is an elongated organ having a length of about 20 cm from right to left. A bulging part on the right side is called a pancreatic head, and a central part is called a pancreatic body, and a thin part on the left side is called a pancreatic tail. The pancreas has an exocrine function of secreting digestive juice called pancreatic juice and an endocrine function of secreting hormones (insulin, glucagon, somatostatin, and the like) which can regulate blood sugar and the amount of pancreatic juice. Diabetes, which is a leading metabolic disease affecting 537 million people worldwide, is caused by abnormalities in the endocrine system of the pancreas. Endocrine glands present in the pancreas are called pancreatic islets, the majority of which are located in the pancreatic tail. Pancreatic islets are composed of α cells which secrete glucagon, β cells which secrete insulin, and δ cells which secrete somatostatin. The βcells are mostly responsible for metabolic abnormalities associated with diabetes. Type 2 diabetes, which accounts for about 95% of all diabetes cases, may be developed by a relative insulin deficiency due to inadequate secretion of insulin, the only blood glucose-lowering hormone in the living body, and insulin resistance. This relative insulin deficiency may occur when pancreatic β cells are exhausted by chronic persistent hyperglycemic conditions, resulting in decrease in the functions and the number of them.

The inventors have been involved in the research and development of anti-diabetic drugs and diabetes preventing ingredients which can target pancreatic β cells. One of the achievements obtained from our research and development demonstrates that methylated flavonoids contained in citrus fruit skin, such as nobiletin, have strong antidiabetic activities by means of their protective effects on pancreatic β cells (Non-Patent Literature 1). With the goal of developing a new curative method of treating diabetes, the present inventors have discovered the DGK δ gene as a factor related to the regeneration and proliferation of reduced p cells due to diabetes (Non-Patent Literature 2). DGK δ is localized in the nuclei of pancreatic β cells, suppressed expression of which may promote the proliferation of pancreatic β cells to ameliorate the pathological conditions of diabetes. Therefore, if a drug which can show protective effects on pancreatic β cells, such as nobiletin, or a drug which can target DGK δ to control the regeneration and proliferation of pancreatic β cells can be delivered into pancreatic islets of the pancreas, a novel curative method for treating diabetes can be achieved without need of transplantation.

### CITATION LIST

### Non Patent Literature

Non Patent Literature 1: Yukiko Kaneko, Toshiyuki Kan, and Tomohisa Ishikawa, “Citrus Flavonoids as a Target for the Prevention of Pancreatic β-cells Dysfunction in Diabetes”, Folia Pharmacologica Japonica, 155(4): 209-213 (2020)
Non Patent Literature 2: Taiji Sato, Chihiro Ishiwatari, Yukiko K. Kaneko et al., “Diacylglycerol kinase δ functions as a proliferation suppressor in pancreatic β-cells”, The FASEB Journal, 35(5): e21420 (2021)

### SUMMARY OF THE INVENTION

### Technical Problem

However, the migration of drugs to the pancreas, in particular to pancreatic islets, via systemic blood circulation by oral administration or injection is in general extremely poor, and thus it is difficult to obtain pharmacological effects on pancreatic β cells present in pancreatic islets by conventional administration methods. Administration of a large amount of a drug may raise some concern about adverse reactions to the liver which is a metabolizing organ albeit potential of pharmacological effects. This is, therefore, impractical.

Meanwhile, lipid nanoparticles (LNPs) are one of the most common drug delivery system (DDS) technologies in practical applications, especially for anticancer drugs. Lipid nanoparticles, which have drug molecules enclosed therein and further have PEG-modified lipids as their constituents, can effectively avoid the reticuloendothelial system (RES), leading to improved pharmacokinetics. This has been one of the useful strategies to reduce toxicity while maintaining the efficacies of chemotherapeutic agents. However, liposome formulations currently in practical use mainly promote accumulation in tumor tissues by improving blood retention and utilizing the EPR effects, and thus specific targeting to an organ other than tumor tissues is difficult. Therefore, a drug delivery technology is desperately needed which can improve migration and accumulation in the pancreas and pancreatic islets where pancreatic β cells reside.

Accordingly, the present invention is made in view of the aforementioned issues. An object of the present invention is to provide lipid nanoparticles enabling excellent drug migration to the pancreas.

### Solution to Problem

After conducting intensive studies about various configurations of lipid nanoparticles in order to achieve targeting of the pancreas with lipid nanoparticles whether the pancreas has neovascularization or not, the inventors found lipid nanoparticles enabling excellent migration and accumulation to the pancreas. The present invention is completed based on this finding.

To solve the above problems, the lipid nanoparticles according to the present invention are lipid nanoparticles for delivering a drug to a target tissue, comprising: (a) a phosphatidylcholine-type phospholipid having an unsaturated fatty acid chain with 16 to 24 carbon atoms, and (b) a polyethylene glycol-modified lipid, wherein the target tissue is the pancreas.

The lipid nanoparticles are composed of a phospholipid and a polyethylene glycol-modified lipid, and (a) the phosphatidylcholine-type phospholipid having an unsaturated fatty acid chain with 16 to 24 carbon atoms is selected as this component phospholipid to improve migration to the pancreas. Hence, a drug can be efficiently delivered to the pancreas by administering the drug contained in these lipid nanoparticles. It is noted that a range of a numerical value shown as “the lower limit to the upper limit” as used herein means that a range of a numerical value shown as “the lower limit or more and the upper limit or less” unless otherwise specified.

In the lipid nanoparticles according to the present invention, (a) the phospholipid as described above may preferably be 1,2-dioleoyl-sn-glycero-3-phosphocholine (DOPC). By this, a compound particularly suitable as (a) the phospholipid can be selected.

It may also be preferred that the lipid nanoparticles according to the present invention have an average particle size of 100 nm to 200 nm, and the target tissue is a pancreatic islet of the pancreas. Lipid nanoparticles enabling selective migration and accumulation of a drug to a pancreatic islet of the pancreas can be obtained by adjusting the average particle size of the lipid nanoparticles to 100 nm to 200 nm.

It may also be preferred that the lipid nanoparticles according to the present invention have an average particle size of 10 nm to 50 nm, and the target tissue is an exocrine tissue of the pancreas. Lipid nanoparticles enabling migration of a drug to an exocrine tissue of the pancreas can be obtained by adjusting the average particle size of the lipid nanoparticles to 10 nm to 50 nm.

It may also be preferred that the lipid nanoparticles according to the present invention further comprise (c) a sterol lipid. By this, lipid nanoparticles enabling improved migration and accumulation to the pancreas can be obtained.

It may also be preferred that in the lipid nanoparticles according to the present invention, relative to the total amount of lipids constituting the lipid nanoparticles, the content percentage of (a) the phospholipid is 40 mol % to 75 mol %, and the content percentage of (b) the polyethylene glycol-modified lipid is 0.01 mol % to 10 mol %, and the content percentage of (c) the sterol lipid is 25 mol % to 60 mol %. By this, the suitable blending percentage of components of the lipid nanoparticles according to the present invention can be selected.

It may also be preferred that in the lipid nanoparticles according to the present invention, (b) the polyethylene glycol-modified lipid is distearoyl-rac-glycerol-PEG (DSG-PEG), 1,2-distearoyl-sn-glycero-3-phosphoethanolamine-N-[amino(polyethylene glycol)](DSPE-PEG), or 1,2-dimyristoyl-rac-glycero-3-methoxypolyethylene glycol (DMG-PEG). By this, a compound suitable as (b) the polyethylene glycol-modified lipid can be selected.

It may also be preferred that in the lipid nanoparticles according to the present invention, (c) the sterol lipid is cholesterol or a cholesterol derivative. By this, a compound suitable as (c) the sterol lipid can be selected.

It may also be preferred that the lipid nanoparticles according to the present invention further comprise (d) an ionized lipid. By this, nucleic acid molecules can be stably retained within the lipid nanoparticles. Therefore, lipid nanoparticles which can deliver nucleic acid as a drug can be obtained when nucleic acid molecules are selected as the drug.

The lipid nanoparticles according to the present invention may further comprise a drug for treating a pancreatic disease. Inclusion of a drug for treating a pancreatic disease in the lipid nanoparticles according to the present invention which will migrate to the pancreas, enables efficient delivery of a therapeutic drug to the pancreas as a target tissue.

A medicament according to the present invention comprises the aforementioned lipid nanoparticles comprising a drug for treating a pancreatic disease as an active ingredient. By this, a medicament can be obtained which can efficiently deliver a therapeutic drug to the pancreas as a target tissue.

### Advantageous Effects of the Invention

The present invention can provide lipid nanoparticles with the following advantageous effects.
(1) can efficiently deliver a drug to the pancreas.
(2) enable a drug to selectively migrate and accumulate to a pancreatic islet of the pancreas when the average particle size of the lipid nanoparticles is configured to be 100 nm to 200 nm.
(3) enable a drug to migrate and accumulate to an exocrine tissue of the pancreas when the average particle size of the lipid nanoparticles is configured to be 10 nm to 50 nm.

### BRIEF DESCRIPTION OF THE DRAWINGS

FIG. 1 shows photographs from ex vivo fluorescence imaging for the distributions of the lipid nanoparticles from Example 1 and Comparative Example 1 to the pancreas and the spleen.

FIG. 2 shows photographs from ex vivo fluorescence imaging for the distributions of lipid nanoparticles from Example 1 and Comparative Example 1 to the liver.

FIG. 3 shows photographs of observation results from fluorescence microscopy on frozen sections prepared from (a) pancreas removed 4 hours after administration of the lipid nanoparticles from Example 1 and (b) pancreas removed 24 hours after administration of the lipid nanoparticles from Example 1.

FIG. 4 shows photographs of observation results from fluorescence microscopy on frozen sections prepared from (a) pancreas removed 4 hours after administration of the lipid nanoparticles from Comparative Example 1 and (b) pancreas removed 24 hours after administration of the lipid nanoparticles from Comparative Example 1.

FIG. 5 shows graphs of brightness spectra from line profile analysis on fluorescence microscopy images on frozen sections prepared from pancreas removed 24 hours after administration of lipid nanoparticles: (a) the data for the lipid nanoparticles from Example 1 and (b) the data for the lipid nanoparticles from Comparative Example 1.

FIG. 6 shows graphs of the distribution of the lipid nanoparticles from Example 1 and Comparative Example 1 in the pancreas.

FIG. 7 shows photographs of observation results of fluorescence microscopy on frozen sections prepared from pancreases removed 1 and 4 hours after administration of lipid nanoparticles: photographs of administration results of (a) the lipid nanoparticles from Example 2 and (b) the lipid nanoparticles from Comparative Example 2.

FIG. 8 shows photographs from ex vivo fluorescence imaging for the distributions of the lipid nanoparticles from Examples 3 and 4 and Comparative Examples 3 and 4 to the pancreas and spleen.

FIG. 9 shows photographs from ex vivo fluorescence imaging for the distributions of the lipid nanoparticles from Examples 3, 4 and Comparative Examples 3, 4 to the liver.

FIG. 10 shows photographs of observation results from fluorescence microscopy on frozen sections prepared from pancreas removed 4 hours after administration of lipid nanoparticles: photographs of administration results of (a) the lipid nanoparticles from Example 3 and Comparative Example 3 and (b) the lipid nanoparticles from Example 4 and Comparative Example 4.

FIG. 11 shows graphs of the distributions of the lipid nanoparticles with different average particle sizes from Examples 1, 3, and 4 in each organ.

FIG. 12 shows graphs of the distributions of the lipid nanoparticles from Example 5 and Comparative Examples 5, 6 in each organ.

FIG. 13 shows photographs from ex vivo fluorescence imaging for the distribution of each type of the lipid nanoparticles from Example 6 to the pancreas and spleen.

FIG. 14 shows photographs from ex vivo fluorescence imaging for the distribution of each type of the lipid nanoparticles from Example 6 to the liver.

FIG. 15 shows photographs of observation results from fluorescence microscopy on frozen sections prepared from pancreas removed 4 hours after administration of lipid nanoparticles: photographs of administration results of (a) the lipid nanoparticles from Example 6-1, (b) the lipid nanoparticles from Example 6-2, (c) the lipid nanoparticles from Example 6-3, and (d) the lipid nanoparticles from Example 6-4.

FIG. 16 shows photographs from ex vivo fluorescence imaging for the distributions of the lipid nanoparticles from Example 9 and Comparative Example 7 to the pancreas and spleen.

FIG. 17 shows photographs from ex vivo fluorescence imaging for the distributions of the lipid nanoparticles from Example 9 and Comparative Example 7 to the liver.

FIG. 18 shows graphs of the distribution in each organ 24 hours after administration of (a) the lipid nanoparticles from Comparative Example 7, (b) the lipid nanoparticles from Example 9-1, (c) the lipid nanoparticles from Example 9-2, and (d) the lipid nanoparticles from Example 9-3.

FIG. 19 shows photographs of observation results from fluorescence microscopy on frozen sections prepared from pancreas removed 24 hours after administration of lipid nanoparticles: photographs of administration results of (a) the lipid nanoparticles from Example 7, (b) the lipid nanoparticles from Example 9-1, (c) the lipid nanoparticles from Example 9-2, and (d) the lipid nanoparticles from Example 9-3.

FIG. 20 shows graphs of measurement results of the anti-apoptotic effects on pancreatic β cells when nobiletin alone or nobiletin-containing lipid nanoparticles were administered in Example 10.

FIG. 21 shows graphs of the gene knockdown effects on pancreatic β cells when siRNA alone, siRNA-containing lipid nanoparticles, or a combination of siRNA and a transfection reagent were used in Example 11.

FIG. 22 shows photographs of the distribution in pancreatic β cells of the lipid nanoparticles according to the present invention by immunohistochemical staining in Example 12.

### DETAILED DESCRIPTION OF THE INVENTION

The lipid nanoparticles according to the present invention will be described in detail below. The lipid nanoparticles according to the present invention are lipid nanoparticles used for delivering a drug to a target tissue, in which the nanoparticles comprise at least (a) a phosphatidylcholine-type phospholipid having an unsaturated fatty acid chain with 16 to 24 carbon atoms, and (b) a polyethylene glycol-modified lipid. The term “lipid nanoparticles” as used in the present invention includes particles having a liposome structure composed of a lipid bilayer as well as particles having a micelle structure composed of a single layer. A drug to be delivered, which is contained in these lipid nanoparticles and delivered to a target tissue, may be contained within the cores of the lipid nanoparticles or between lipid bilayers of the lipid nanoparticles, depending on the degree of hydrophilicity of the drug.

The target tissue for drug transport of the lipid nanoparticles according to the present invention is the pancreas. In addition, the lipid nanoparticles according to the present invention can selectively migrate to a pancreatic islet or an exocrine tissue of the pancreas by adjusting their particle sizes within a predetermined range as described below. Specifically, when the average particle size of the lipid nanoparticles is 100 to 200 nm, migration and accumulation to a pancreatic islet of the pancreas can be enhanced, and the pancreatic islet of the pancreas can be considered as a target tissue. When the average particle size of the lipid nanoparticles is 10 to 50 nm, migration and accumulation to an exocrine tissue of the pancreas can be enhanced, and the exocrine tissue of the pancreas can be considered as a target tissue. This means, for example, that when drug delivery to a pancreatic islet is desired for treating diabetes or other diseases, lipid nanoparticles may be sized to 100 to 200 nm, and when drug delivery to a pancreatic exocrine tissue is desired for treating pancreatic cancer or other diseases, lipid nanoparticles may be sized to 10 to 50 nm.

Next, each of the components constituting the lipid nanoparticles according to the present invention will be described. A neutral glycerophospholipid is used as a phospholipid, and (a) a phosphatidylcholine-type phospholipid having an unsaturated fatty acid chain with 16 to 24 carbon atoms is included in the lipid nanoparticles according to the present invention. At least one of the two fatty acids attached to a glycerin moiety may be an unsaturated fatty acid, but preferably both of them are unsaturated fatty acids. The number of carbons of the two fatty acids attached to the glycerol moiety may preferably be 16 to 24, more preferably 17 to 23, and in particular preferably 17 to 19. More specifically, examples of (a) the phospholipid include in particular preferably 1,2-dioleoyl-sn-glycero-3-phosphocholine (DOPC) used in Examples described below. Use of this DOPC as a phospholipid constituting the lipid nanoparticles can reduce migration and accumulation to the liver and spleen, and can enhance migration and accumulation to the pancreas.

The lipid nanoparticles according to the present invention also comprise (b) a polyethylene glycol-modified lipid. In general, (b) the polyethylene glycol-modified lipid may be any polyethylene glycol-modified lipid as long as it is used as a component of lipid nanoparticles for drug delivery, including, but not limited to, DMG-PEG, ALC-0159, DSPE-PEG, DSG-PEG, and the like. Among these, distearoyl-rac-glycerol-PEG (DSG-PEG) in which glyceryl distearate (DSG) is PEGylated, and DSPE-PEG in which 1,2-distearoyl-sn-glycero-3-phosphoethanolamine (DSPE) is PEGylated, and 1,2-dimyristoyl-rac-glycero-3-methoxypolyethylene glycol (DMG-PEG) are preferably used. The molecular weight of added polyethylene glycol is preferably 500 to 10000 in terms of the number average molecular weight (Mn), more preferably 1000 to 5000, and in particular preferably 1000 to 3000. It is noted that the number average molecular weight (Mn) as used herein refers to a value in terms of standard polyethylene glycol obtained by gel permeation chromatography (GPC).

For the blending percentages of the aforementioned components (a) and (b) in the lipid nanoparticles according to the present invention, the content percentage of (a) the phospholipid relative to the total amount of lipids constituting the lipid nanoparticles is preferably 90 mol % to 99.99 mol %, more preferably 90 mol % to 99.5 mol %, and even more preferably 92 mol % to 99 mol %. For the (b) polyethylene glycol-modified lipid, the content percentage of (b) the polyethylene glycol-modified lipid relative to the total amount of lipids constituting the lipid nanoparticles is preferably 0.01 mol % to 10 mol %, more preferably 0.5 mol % to 10 mol %, and even more preferably 1 mol % to 8 mol %.

The lipid nanoparticles according to the invention may also comprise (c) a sterol lipid. This can enhance migration and accumulation to the pancreas. In general, (c) the sterol lipid may be any sterol lipid as long as it is used as a component of lipid nanoparticles for drug delivery, including, but not limited to, cholesterol, as well as cholesterol derivatives such as dihydrocholesterol and cholesterol esters, campesterol, and phytosterols such as sitosterol and stigmasterol, and the like. Among these, cholesterol or cholesterol derivatives are preferred. Cholesterol derivatives include cholesteryl hemisuccinate (CHEMS) and the like.

For the blending percentages of the aforementioned components (a) to (c) in the lipid nanoparticles according to the present invention, the content percentage of (a) the phospholipid relative to the total amount of lipids constituting the lipid nanoparticles is preferably 40 mol % to 75 mol %, more preferably 45 mol % to 70 mol %, and even more preferably 50 mol % to 65 mol %. For (c) the sterol lipid, the content percentage of (c) the sterol lipid relative to the total amount of lipids constituting the lipid nanoparticles is preferably 25 mol % to 60 mol %, more preferably 30 mol % to 55 mol %, and even more preferably 35 mol % to 50 mol %. For the (b) polyethylene glycol-modified lipid, the content percentage of (b) the polyethylene glycol-modified lipid relative to the total amount of lipids constituting the lipid nanoparticles is preferably 0.01 mol % to 10 mol %, more preferably 0.5 mol % to 10 mol %, and even more preferably 1 mol % to 8 mol %.

The lipid nanoparticles according to the present invention may further comprise (d) an ionized lipid in addition to the aforementioned lipid components (a) to (c). In a case that a nucleic acid molecule is selected as a drug, the nucleic acid molecule can be stably enclosed in the lipid nanoparticles while increasing migration and accumulation to the pancreas. This can be achieved because the nucleic acid molecule can be held stably within the lipid nanoparticles by virtue of the electrostatic interaction between the ionized lipid and the nucleic acid molecule. As (d) the ionized lipid, any known ionized lipids can be used as a component of the lipid nanoparticles for delivering a nucleic acid molecule, including those commercialized or used in basic research and the like. Specific examples include, but not limited to, for example, D-Lin-MC3-DMA, SM-102, ALC-0315, cKK-E12, D-Lin-KC2-DMA, DODAP, DODMA, DOTAP, 93-0170, 93-017S, 3060i10, L-319, C12-200, LPO1, Lipid5, Lipid29, ssPalm-OP, ssPalm-E, ssPalm-EC, ssPalm-OC, and the like.

For the blending percentages of the aforementioned components (a) to (d) in the lipid nanoparticles according to the present invention, the content percentage of (d) the ionized lipid relative to the total amount of lipids constituting the lipid nanoparticles is preferably 30 mol % to 60 mol %, more preferably 35 mol % to 55 mol %, and even more preferably 40 mol % to 50 mol %. For (a) the phospholipid, the content percentage of (a) the phospholipid relative to the total amount of lipids constituting the lipid nanoparticles is preferably 10 mol % to 30 mol %, more preferably 10 mol % to 25 mol %, and even more preferably 15 mol % to 25 mol %. For (c) the sterol lipid, the content percentage of (c) the sterol lipid relative to the total amount of lipids constituting the lipid nanoparticles is preferably 20 mol % to 45 mol %, more preferably 25 mol % to 40 mol %, and even more preferably 25 mol % to 35 mol %. For (b) the polyethylene glycol-modified lipid, the content percentage of (b) the polyethylene glycol-modified lipid relative to the total amount of lipids constituting the lipid nanoparticles is preferably 0.01 mol % to 10 mol %, more preferably 0.5 mol % to 10 mol %, and even more preferably 1 mol % to 8 mol.

It is noted that a lipid component other than the aforementioned lipid components (a) to (d) may also be blended in the lipid nanoparticles according to the present invention, as needed.

A drug to be delivered to the pancreas as a target tissue is contained in the lipid nanoparticles according to the present invention. The term “drug” as used in the present invention refers to a concept including low molecular weight compounds, peptides (such as bioactive peptides, hormone-like peptides, cytokine-like peptides, cyclic peptides, and synthetic peptides), proteins (such as antibodies, enzymes, nutritional factors, cytokines, and hormones), nucleic acids (such as plasmid DNAs, siRNAs, miRNAs, antisense nucleic acids, shRNAs, pre-miRNAs, pri-miRNAs, mRNAs, decoy nucleic acids, ribozymes, DNA aptamers, RNA aptamers, DNA enzymes, and the like), lipids, and the like. Drugs for treating pancreatic diseases include, but not limited to, drugs for treating diabetes by targeting pancreatic islets, and drugs for treating pancreatic cancer and pancreatitis by targeting pancreatic exocrine tissues. Drugs for treating diabetes by targeting pancreatic islets include, for example, methylated flavonoids contained in citrus fruit skin such as nobiletin, nucleic acid medicaments for inhibiting expression of DGK δ (e.g., siRNA, miRNA, or the like), and the like. Drugs for treating pancreatic cancer and pancreatitis by targeting pancreatic exocrine tissues include, for example, anticancer agents such as doxorubicin, protease inhibitors such as Foipan, and the like.

The content of a drug contained in the lipid nanoparticles may appropriately be selected according to the type of the drug and the size (particle size) of the lipid nanoparticles themselves. For example, it is drug/total lipid components constituting the lipid nanoparticles=0.01 to 1 (by mass ratio), but not limited to this.

The average particle size of the lipid nanoparticles according to the present invention is preferably 500 nm or less, more preferably 300 nm or less. The lipid nanoparticles according to the present invention, which can efficiently migrate and accumulate to the pancreas, can further target a pancreatic islet or an exocrine tissue in the pancreas by adjusting their particle size within a predetermined range. In a case where a pancreatic islet of the pancreas is selected as a target tissue, the average particle size of the lipid nanoparticles is preferably 80 to 200 nm, more preferably 100 to 200 nm, and in particular preferably 120 to 180 nm. Migration and accumulation of a drug in a pancreatic islet of the pancreas can be enhanced by adjusting the average particle size of the lipid nanoparticles to these ranges. On the other hand, in a case where an exocrine tissue of the pancreas is selected as a target tissue, the average particle size of the lipid nanoparticles is preferably between 10 nm and 80 nm, more preferably 10 nm to 50 nm, and in particular preferably 20 nm to 50 nm. Migration and accumulation of a drug in an exocrine tissue of the pancreas can be enhanced by adjusting the average particle size of the lipid nanoparticles to these ranges. It is noted that the term “average particle size” as used herein refers to an average particle size determined by dynamic light scattering, which can be measured using a light scattering particle size analyzer (e.g., Zetasizer Ultra available from Malvern Panalytical Ltd.). Light scattering particle size analyzers can measure a cumulant average particle size and a mass average particle size, and a cumulant average particle size as measured by a Zetasizer Ultra from Malvern Panalytical Ltd. may preferably be used.

The method of manufacturing a lipid nanoparticle according to the present invention will be described. The lipid nanoparticles according to the present invention can be obtained by dissolving or dispersing lipid components including at least (a) a phospholipid and (b) a polyethylene glycol-modified lipid in an organic solvent to prepare a lipid solution, which is then mixed and stirred with an aqueous solution. The lipid components may also include (c) a sterol lipid and (d) an ionized lipid. Here, a drug to be contained in the lipid nanoparticles can be dissolved or dispersed in the lipid solution along with the lipid components, or can be dissolved or dispersed in an aqueous solution. It is noted that alcohols such as ethanol and methanol, ethers such as THF, acetone, acetonitrile, and the like can be used as an organic solvent used in the lipid solution. Buffer solutions (such as PBS and HEPES), physiological saline, and the like can be used as the aqueous solution in addition to water as it is. When mixing a lipid solution including lipid components with an aqueous solution, any mixing methods which are known to be used in methods of manufacturing lipid nanoparticles can be used. Examples include an organic solvent injection method, in which a lipid solution is injected into an aqueous solution with a syringe and then stirred; a microfluidic device method, in which a lipid solution is mixed with an aqueous solution in a mixing channel having a special channel structure; a thin film hydration method, in which an organic solvent is removed from a lipid solution to form a lipid film, and the lipid film is hydrated with an aqueous solution; and the like. In the present invention, the microfluidic device method is preferably used for manufacturing lipid nanoparticles in view that the average particle size of the resulting lipid nanoparticles can be precisely controlled. In particular, a dispersion containing the lipid nanoparticles according to the present invention can be easily obtained by using a nanoparticle manufacturing device (a nanoparticle manufacturing platform LiNAS-M, from Lilac pharma Inc.) equipped with an iLiNP microfluidic chip (Lilac pharma Inc.). Specifically, a dispersion containing the lipid nanoparticles according to the present invention can be manufactured by mixing an aqueous solution (PBS) containing a drug with a lipid solution (ethanol solution) in an iLiNP microfluidic chip. The average particle size of the resulting lipid nanoparticles can be controlled by varying the total lipid concentration in the lipid solution, and/or the total flow rate (TFR) and/or the flow rate ratio (FRR) of the lipid solution and the aqueous solution. The dispersion manufactured by this method includes the lipid nanoparticles and the solvents (e.g., PBS and ethanol) for the aqueous solution and the lipid solution. The dispersion can be concentrated by removing and/or replacing the solvents using dialysis, ultrafiltration, or other processes.

In addition to the components described above, the lipid nanoparticles ac cording to the present invention can also comprise a pharmacologically acceptable e carrier for common formulations and an additive acceptable as an additive for pharmaceutical products.

The medicament according to the present invention comprises lipid nanoparticles containing a drug for treating a pancreatic disease as an active ingredient. This can be used to prevent, ameliorate, or treat pathological conditions, symptoms, or diseases involving pancreatic injury, dysfunction, and the like. Treatment and amelioration of pathological conditions as used herein also include preventive and aftercare treatments against pancreatic diseases and pathological conditions. Pancreatic diseases include diabetes, pancreatitis, pancreatic cancer, and the like or related symptoms or diseases thereof. The medicament having the lipid nanoparticles according to the present invention as an active ingredient may be used to treat or ameliorate one or more of these pathological conditions, symptoms, or diseases, or to suppress or prevent symptoms or pathological conditions. The medicament according to the present invention can be used as a quasi-drug or a food product other than a medicament for human or animal. Food products include supplements, health foods, functional foods, foods for specified health uses, and the like.

The lipid nanoparticles according to the present invention may be used mainly in a form of injectable and infusion agents. It can be used in a form of inhalation such as powder and aerosol, and it can also be used in a form of transdermal absorbent such as patch, suppository, and the like when used as a parenteral agent such as a topical agent. Furthermore, when used as an oral formulation, it can be used in a form of a tablet, a granule, a capsule, or an oral liquid. There is no particular limitation for the dosage or effective intake of a medicament having the lipid nanoparticles according to the present invention as an active ingredient because it varies depending on the content and quantity of a drug to be contained in the lipid nanoparticles, intended therapeutic effects, administration methods, administration targets, and dosage forms.

### EXAMPLES

Although the present invention will be specifically described below with reference to Examples and Comparative Examples, the present invention is not particularly limited to these Examples. Methods of preparing lipid nanoparticles and methods of measuring physical properties thereof in the following Examples and Comparative Examples are as follows.

### (1) Components of the Lipid Nanoparticles

Compounds used as components of the lipid nanoparticles prepared in Examples and Comparative Examples are listed below.

### (a) Phospholipids:

1,2-dioleoyl-sn-glycero-3-phosphocholine (hereinafter also referred to as “DOPC”) from Avanti Polar Lipids, LLC.
1,2-distearoyl-sn-glycero-3-phosphocholine (hereinafter also referred to as “DSPC”) from Avanti Polar Lipids, LLC.
1,2-dioleoyl-sn-glycero-3-phosphoethanolamine (hereinafter also referred to as “DOPE”) from Avanti Polar Lipids, LLC.

### (b) PEG-Modified Lipids:

distearoyl-rac-glycerol-PEG 2K (the average molecular weight of added polyethylene glycol: 2000, hereinafter also referred to as “DSG-PEG 2000”) from Avanti Polar Lipids, LLC.
1,2-distearoyl-sn-glycero-3-phosphoethanolamine-N-[amino(polyethylene glycol)-2000](ammonium salt) (the average molecular weight of added polyethylene glycol: 2000, hereinafter also referred to as “DSPE-PEG 2000”) from Avanti Polar Lipids, LLC.
1,2-dimyristoyl-rac-glycero-3-methoxypolyethylene glycol-2000) (the average molecular weight of added polyethylene glycol: 2000, hereinafter also referred to as “DMG-PEG 2000”) from Avanti Polar Lipids, LLC.

### (c) Sterol Lipids:

cholesterol (hereinafter also referred to as “Chol”) from Avanti Polar Lipids, LLC.
cholesteryl hemisuccinate (hereinafter also referred to as “CHEMS”) from Avanti Polar Lipids, LLC.

### (d) Ionized Lipids:

D-Lin-MC3-DMA (hereinafter also referred to as “MC3”) from MedChemExpress, LLC.

As a fluorescent label for each of the lipid nanoparticles prepared in Examples and Comparative Examples, used was red fluorescent DiD (1,1′-dioctadecyl-3,3,3′,3′-tetramethylindodicarbocyanine, 4-chlorobenzenesulfonate) from Thermo Fisher Scientific Inc.

### (2) Preparation of Lipid Nanoparticles

The methods of preparing each of the lipid nanoparticles according to Examples 1, 3 to 12, and Comparative Examples 1, 3 to 7 are shown below. Each of the lipid nanoparticles were prepared by a microfluidic device method using an iLiNP (Lilac pharma Inc.), a lipid nanoparticle manufacturing device. Specifically, first, powders of a phospholipid, a sterol lipid, and a PEG-modified lipid, which are the components of lipid nanoparticles, were each dissolved in ethanol to prepare stock solutions. They were stored in glass vials at or below −20° C. until time of use. A sample solution was prepared as follows. Each of the stock solutions was placed in a 1.5 mL plastic tube to give a predetermined molar ratio, and mixed in that tube, and then diluted with ethanol to adjust the concentrations of lipids. It is noted that In Examples 1, 3 to 6, 9, and 12 and Comparative Examples 1, 3 to 7, DiD was added to this sample solution in an amount equivalent to 1 mol % relative to the total mol amount of the lipid components in order to fluorescently label lipid nanoparticles. In order to obtain a lipid nanoparticle-containing dispersion, the sample solution was mixed with a water-soluble solution using a nanoparticle manufacturing device (a nanoparticle manufacturing platform LiNAS-M from Lilac pharma, Inc.). It is noted that a PBS solution was used for the water-soluble solution. To obtain uniform particle sizes, preparation was performed by varying conditions within the ranges where the total lipid concentration in the sample solution was 10 to 25 mM, and the total flow rate (TFR) was 150 to 2000 mL/min, and the flow rate ratio (FRR) was 1 to 3 (water-soluble solution/sample solution). The resulting lipid nanoparticle-containing dispersion was dialyzed against a PBS solution (pH 7.4) for 2 hours or more using a dialysis membrane (Spectra/Por® 4 from REPLIGEN Corporation) with a molecular weight cut off of 12000 to 14000. The lipid nanoparticle dispersion was then concentrated using a centrifugal filtration device (Macrosep 3K, 4 mL, Nihon Pall Ltd.) at 20° C. and 4700 G to obtain a final concentration of lipid nanoparticles of 25 mM. The average particle size (the cumulant average particle size by dynamic light scattering), the polydispersity index (PDI), and the zeta potential (particle charge) of the resulting lipid nanoparticles were measured with a light scattering analyzer (Zetasizer Ultra from Malvern Panalytical Ltd.). The preparation of the lipid nanoparticles in Example 2 and Comparative Example 2 was performed by a thin-film hydration method.

### Example 1

### 1-1. Intravenous Administration of Lipid Nanoparticles (Phospholipid: DOPC, Average Particle Size: 166 nm) and Organ Isolation and Investigation on Intraorgan Distribution by Fluorescence Imaging Observation

In this Example, 1,2-dioleoyl-sn-glycero-3-phosphocholine (DOPC) was used as a phospholipid, and cholesterol (Chol) was used as a sterol lipid, and distearoyl-rac-glycerol-PEG 2K (DSG-PEG 2000) was used as a PEG-modified lipid. Using a composition of DOPC:Chol:DSG-PEG 2000=60:40:5 (by molar ratio), the total lipid concentration of the sample solution to be pumped into a microfluidic device (final concentration: 25 mM), the total flow rate of the sample solution and the water-soluble solution (TFR=250 mL/min), and the flow rate ratio (FRR=1) were adjusted so as to obtain lipid nanoparticles with an average particle size of about 150 nm to 200 nm, thereby obtaining the lipid nanoparticles according to Example 1. Table 1 below shows the average particle size and PDI of the resulting lipid nanoparticles.

Animal experiments were conducted in accordance with the protocols approved based on the regulations on animal care and use at University of Shizuoka. The lipid nanoparticles were administered to 8-week old male C57BL/6J mice (Japan SLC Inc.). The C57BL/6J mice were administered in tail vein with 200 μL (total amount of lipids: 5 mol/mouse) of the dispersion of DiD-labeled lipid nanoparticles prepared in this Example. After 1, 4, and 24 hours, each mouse was anesthetized by inhalation of isoflurane, and blood was then collected from the inferior vena cava, and the heart, lung, pancreas, spleen, liver, kidney, and gastrocnemius and soleus muscles were isolated. In addition, a vascular labeling reagent, DyLight488-labeled lectin (Lycopersicon esculentum (Tomato) Lectin-LEA, DyLight488, from Thermo Fisher Scientific, Inc.) was diluted 4-fold in PBS, 200 μL (50 g in terms of LEL) of which was then administered in tail vein to some of the mice 5 minutes before anesthetization.

All of the isolated organs were observed using a fluorescence imaging system (IVIS Lumina III, from Summit Pharmaceuticals International Corporation) at Ex/Em: 644 nm/663 nm, and the resulting fluorescence imaging images were ROI-analyzed to quantify the fluorescence intensity of DiD (indicating the distribution of the lipid nanoparticles) in each organ.

Frozen specimens of the isolated pancreas were also prepared. The isolated pancreas was impregnated with 4% fetal bovine serum (FBS) and allowed to stand overnight, and then impregnated with 10% sucrose for 4 hours, followed by 15% sucrose for 4 hours. It was then impregnated with 20% sucrose and allowed to stand overnight. Into a Cryomold, added and mixed were 300 μL of PBS and an O.C.T. Compound, to which the pancreas was then embedded. This was then frozen in liquid nitrogen and stored at −80° C.

The frozen pancreatic specimen prepared was thinly sliced at a thickness of 5 m in a cryostat, which was then attached to a poly-L-lysine-coated glass slide. This was sealed with a nuclear staining sealant (DAPI-Fluoromount-G® from Cosmo Bio Co., Ltd.). The slide was observed and analyzed with a fluorescence microscope (BZ-X810 from Keyence Corporation). From the captured images, pancreatic islets, exocrine tissues, and vascular areas were designated, and the average brightness of these areas was then analyzed to quantify DiD (indicating the distribution of lipid nanoparticles) and DyLight 488-labeled lectin (indicating vascular areas).

### Comparative Example 1

### 1-2. Intravenous Administration of Lipid Nanoparticles (Phospholipid: DSPC, Average Particle Size: 165 nm) and Organ Isolation and Investigation on Intraorgan Distribution by Fluorescence Imaging Observation

In this Comparative Example, lipid nanoparticles according to Comparative Example 1 were prepared and tested by intra-tail vein administration to mice using similar materials and methods as in Example 1 except that 1,2-distearoyl-sn-glycero-3-phosphocholine (DSPC) was used in place of DOPC which was used as a phospholipid in Example 1, and the total lipid concentration of the sample solution to be pumped into the microfluidic device (final concentration: 25 mM), and the total flow rate (TFR=250 mL/min) and the flow rate ratio (FRR=2) of the sample solution and the water-soluble solution were used. Table 1 below shows the average particle size and PDI of the resulting lipid nanoparticles.

TABLE 1

| | Phospho- | | PEG-modified | Average | |
|---|---|---|---|---|---|
| | lipid | Sterol lipid | lipid | particle | |
| | (molar ratio) | (molar ratio) | (molar ratio) | size | PDI |
| Example 1 | DOPC | Cholesterol | DSG-PEG2000 | 166 nm | 0.259 |
| | (60 mol) | (40 mol) | (5 mol) | | |
| Comparative | DSPC | Cholesterol | DSG-PEG2000 | 165 nm | 0.471 |
| Example 1 | (60 mol) | (40 mol) | (5 mol) | | |

### Results from Example 1 and Comparative Example 1

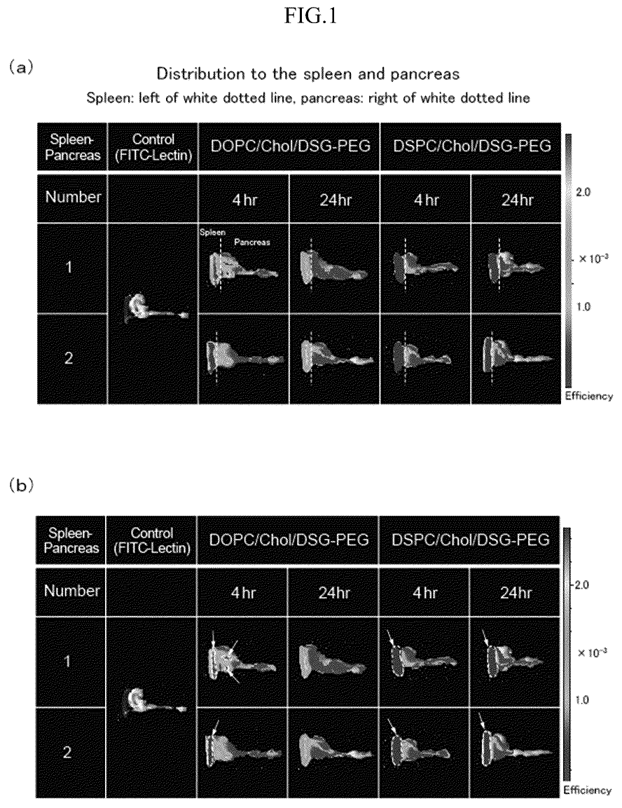

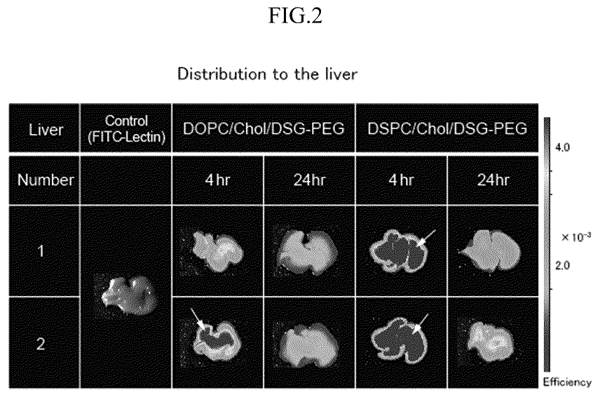

FIG. 1 shows results from ex vivo fluorescence imaging of pancreas and spleen removed 4 and 24 hours after the DiD-labeled lipid nanoparticles prepared in Example 1 and Comparative Example 1 were administered in tail vein to mice. White dotted lines in FIG. 1(a) indicate the boundaries between the spleen (left of the white dotted lines) and pancreas (right of the white dotted lines). In FIG. 1(b), the areas where the fluorescence intensity of DiD is more than 2.2×10⁻³, i.e., the high accumulation areas of lipid nanoparticles, are indicated by white dotted lines and white arrows. FIG. 2 shows results from ex vivo fluorescence imaging of liver removed 4 and 24 hours after the DiD-labeled lipid nanoparticles prepared in Example 1 and Comparative Example 1 were administered in tail vein to mice. With regard to white arrows in FIG. 2, the areas where the fluorescence intensity of DiD is more than 4.0×10⁻³ are indicated by white arrows. These results reveal that the majority of the lipid nanoparticles comprising DSPC according to Comparative Example 1 accumulated in the liver and spleen whereas the lipid nanoparticles comprising DOPC according to Example 1 showed weak fluorescence in the liver and spleen and accumulated most in the pancreas.

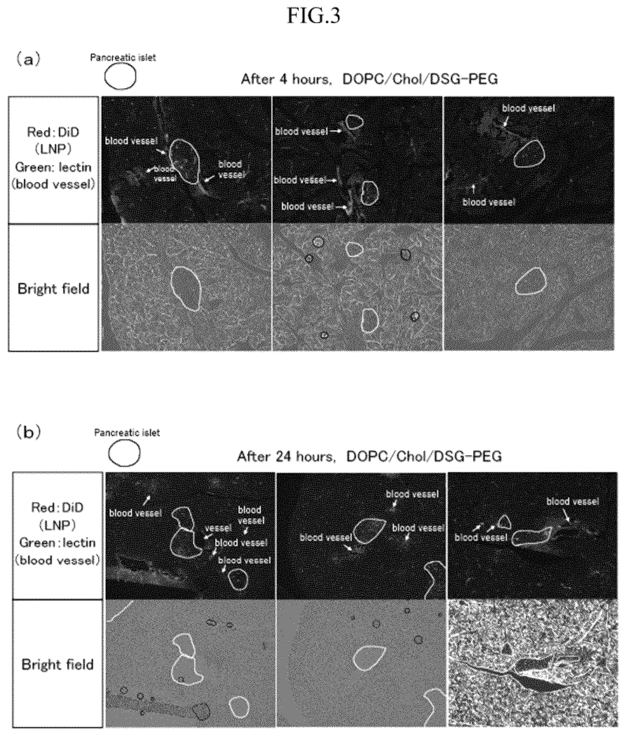

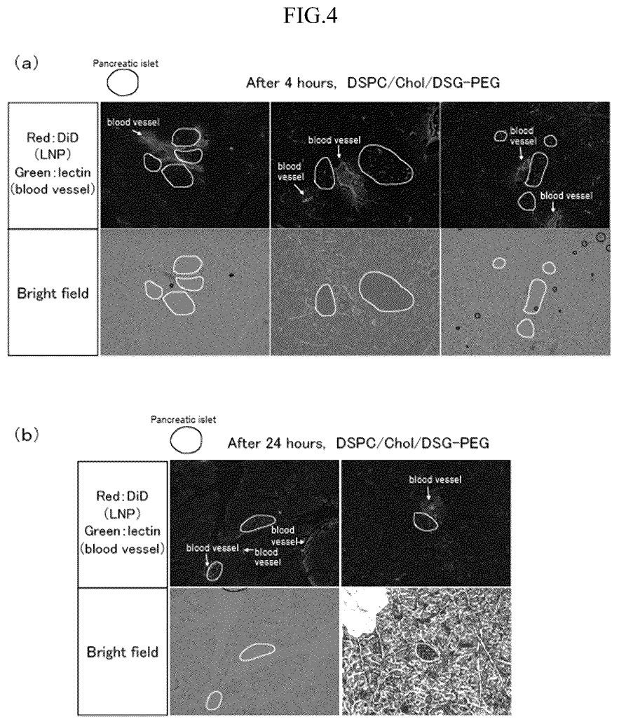

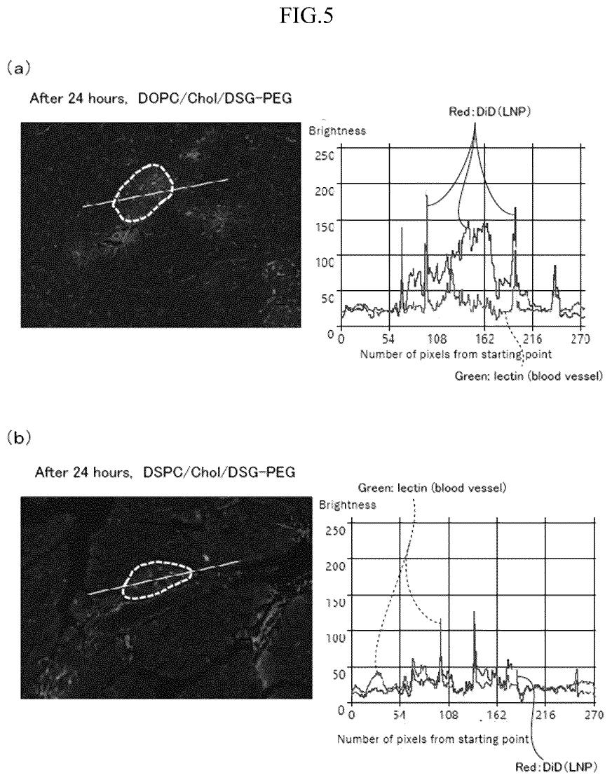

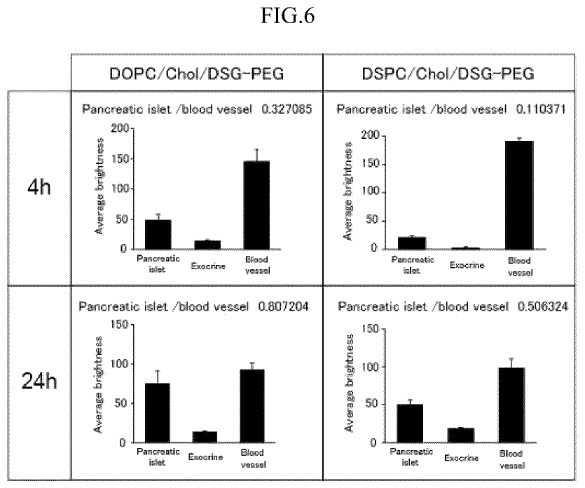

Next, the observation results from fluorescence microscopy on frozen sections prepared from pancreases removed after 4 and 24 hours are shown in FIG. 3 (Example 1) and FIG. 4 (Comparative Example 1). In each of the photographs, white lines indicate pancreatic islets, and white arrows indicate vascular areas. These results reveal that the lipid nanoparticles comprising DSPC according to Comparative Example 1 were mostly distributed in blood vessels and did not migrate to pancreatic islets (FIG. 4) whereas the lipid nanoparticles comprising DOPC according to Example 1 showed a significant red fluorescence due to DiD in pancreatic islets surrounded by white lines, indicating significant migration and accumulation to pancreatic islets. This is supported by the results from line profile analysis as shown in FIG. 5. The data shown in FIG. 5 show the fluorescence brightness in each spectrum of DiD and DyLight 488-labeled lectins on a line (shown as a white line in each photograph) drawn to cross the area where red fluorescence due to DiD was observed (shown by a white dotted line in each photograph) for the photographic data from fluorescence microscopy on the frozen sections prepared from pancreas removed after 24 hours in FIG. 3 (Example 1) and FIG. 4 (Comparative Example 1). These results show that the spectrum of lectin, which indicates vascular areas, does not coincide with the spectrum of DiD, which indicates the distribution of lipid nanoparticles, in a case of the lipid nanoparticles comprising DOPC according to Example 1 in FIG. 5(a). This indicates that the lipid nanoparticles comprising DOPC did not stay in pancreatic vessels, but migrated and accumulated to pancreatic islets. In contrast, the spectrum of lectin, which indicates vascular areas, almost coincides with the spectrum of DiD, which indicates the distribution of lipid nanoparticles, in a case of the lipid nanoparticles comprising DSPC according to Comparative Example 1 in FIG. 5(b). This indicates that the lipid nanoparticles remained in the blood vessels and did not migrate to the tissue. FIG. 6 shows results from analysis of the average brightness of pancreatic islets, exocrine tissues, and vascular areas designated in the captured images of the frozen sections of the pancreas. These results demonstrate that the lipid nanoparticles comprising DOPC according to Example 1 showed excellent distribution to pancreatic islets than the lipid nanoparticles according to Comparative Example 1, indicating excellent migration and accumulation properties to pancreatic islets.

### Example 2

### 2-1. Intravenous Administration of Lipid Nanoparticles (Phospholipid: DOPC, Average Particle Size: 164 nm) and Investigation on Intrapancreatic Distribution

In this Example, lipid nanoparticles were prepared by the thin-film hydration method. In this Example, DOPC was used as a phospholipid, and cholesterol (Chol) was used as a sterol lipid, and DSPE-PEG 2000 was used as a PEG-modified lipid. A lipid solution was prepared as follows. Each of the stock solutions of the phospholipid, the sterol lipid, and the PEG-modified lipid was placed in a tube to give a composition of DOPC:Chol:DSPE-PEG 2000=60:40:5 (by molar ratio), and mixed in that tube, and then diluted with ethanol to obtain a final lipid concentration of 25 mM. DiD in an amount equivalent to 1 mol % relative to the total mol amount of lipid components was added to this lipid solution to fluorescently label the lipid nanoparticles. The solvent was removed from the lipid solution using an evaporator to form a thin lipid film on the flask wall. PBS was poured into the flask as a water-soluble solution to rehydrate this thin lipid film. It was heated in a 37° C. warm bath for 1 minute, and then stirred with a vortex mixer for 1 minute. This was repeated total three times to prepare a lipid nanoparticle-containing dispersion. The average particle size (the cumulant average particle size by dynamic light scattering), the polydispersity index (PDI), and the zeta potential (particle charge) of the resulting lipid nanoparticles were measured with a light scattering analyzer (Zetasizer Ultra from Malvern Panalytical Ltd.). Table 2 below shows the average particle size, PDI, and zeta potential of the resulting lipid nanoparticles. The lipid nanoparticles according to Example 2 was tested by intra-tail vein administration to mice using similar materials and methods as in Example 1 described above. The dosage of the lipid nanoparticles to mice was 200 μL (total amount of lipid: mol/mouse).

### Comparative Example 2

### 2-2. Intravenous Administration of Lipid Nanoparticles (Phospholipid: DOPE, Average Particle Size: 170 nm) and Investigation on Intrapancreatic Distribution

In this Comparative Example, 1,2-dioleoyl-sn-glycero-3-phosphoethanolamine (DOPE) was used in place of DOPC which was used as a phospholipid in Example 2, and cholesteryl hemisuccinate (CHEMS) which can form a stable bilayer with DOPE was used in place of cholesterol which was used as a sterol lipid in Example 2. The lipid nanoparticles according to Comparative Example 2 were prepared and tested by intra-tail vein administration to mice using similar materials and methods as in Example 2 except for these. Table 2 below shows the average particle size, PDI, and zeta potential of the resulting lipid nanoparticles.

TABLE 2

| | Phospholipid | Sterol lipid | PEG-modified lipid | Average | | Zeta | |
|---|---|---|---|---|---|---|---|
| | (molar ratio) | (molar ratio) | (molar ratio) | particle size | PDI | potential | |
| Example 2 | DOPC | Cholesterol | DSPE-PEG2000 | 164 nm | 0.671 | −1.2 | mV |
| | (60 mol) | (40 mol) | (5 mol) | | | | |
| Comparative | DOPE | cholesteryl | DSPE-PEG2000 | 170 nm | 0.384 | −12.6 | mV |

| | | | |
|---|---|---|---|
| Example 2 | (60 mol) | hemisuccinate | (5 mol) |
| | | (40 mol) | |

### Results from Example 2 and Comparative Example 2

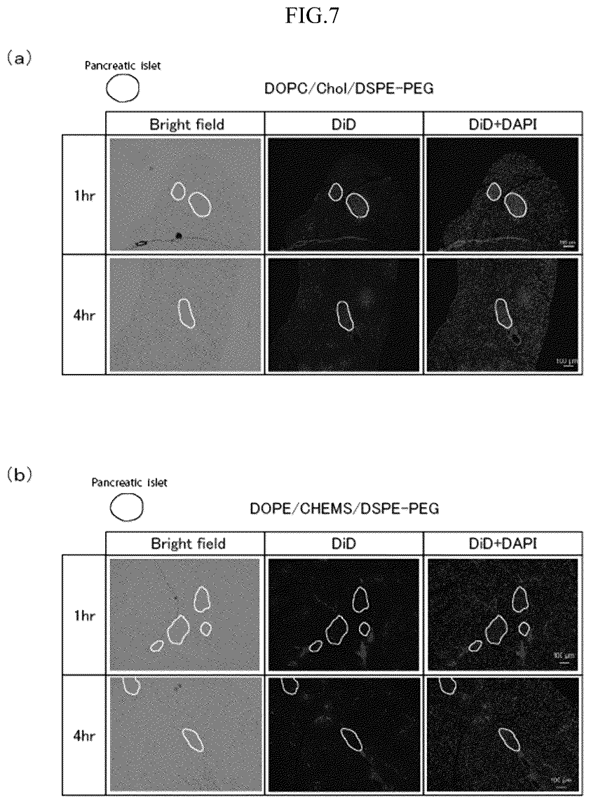

FIG. 7 shows observation results from fluorescence microscopy on frozen sections prepared from pancreases removed 1 and 4 hours after the corresponding lipid nanoparticles were administered in tail vein to mice. White lines in each of the photographs indicate pancreatic islets. FIG. 7(a) shows results from Example 2 and FIG. 7(b) shows results from Comparative Example 2. These results reveal that the lipid nanoparticles comprising DOPE according to Comparative Example 2 showed neither migration nor accumulation to pancreatic islets whereas the lipid nanoparticles comprising DOPC according to Example 2 showed significant red fluorescence due to DiD within pancreatic islets surrounded by white lines, indicating significant migration and accumulation to pancreatic islets as in Example 1 described above.

### Example 3

### 3-1. Intravenous Administration of Lipid Nanoparticles (Phospholipid: DOPC, Average Particle Size: 121 nm) and Organ Isolation and Investigation on Intraorgan Distribution by Fluorescence Imaging Observation

In this Example, the total lipid concentration of the sample solution to be pumped into the microfluidic device (final concentration 25 mM), and the total flow rate (TFR=250 mL/min) and the flow rate ratio (FRR=3) of the sample solution and the water-soluble solution were adjusted so as to obtain lipid nanoparticles with an average particle size of about 80 nm to 130 nm using a similar composition as in Example 1 described above, thereby obtaining the lipid nanoparticles according to Example 3. Table 3 below shows the average particle size and PDI of the resulting lipid nanoparticles. The lipid nanoparticles according to Example 3 were tested by intra-tail vein administration to mice using similar materials and methods as in Example 1. The dosage of the lipid nanoparticles to mice was 200 μL (total amount of lipids: 5 mol/mouse).

### Comparative Example 3

### 3-2. Intravenous Administration of Lipid Nanoparticles (Phospholipid: DOPE, Average Particle Size: 107 nm) and Organ Isolation and Investigation on Intraorgan Distribution by Fluorescence Imaging Observation

In this Comparative Example, the lipid nanoparticles according to Comparative Example 3 were prepared and tested by intra-tail vein administration to mice using similar materials and methods as in Example 3 except that DOPE and CHEMS were used in place of DOPC which was used as a phospholipid, and cholesterol in Example 3. Table 3 below shows the average particle size and PDI of the resulting lipid nanoparticles.

### Example 4

### 4-1. Intravenous Administration of Lipid Nanoparticles (Phospholipid: DOPC, Average Particle Size: 30 nm) and Organ Isolation and Investigation on Intraorgan Distribution by Fluorescence Imaging Observation

In this Example, using a similar composition as in Example 1 described above, the total lipid concentration of the sample solution to be pumped into the microfluidic device (final concentration: 25 mM), and the total flow rate (TFR=2000 mL/min) and the flow rate ratio (FRR=3) of the sample solution and the water-soluble solution were adjusted so as to obtain lipid nanoparticles with an average particle size of about 20 nm to 60 nm, thereby obtaining the lipid nanoparticles according to Example 4. Table 3 below shows the average particle size and PDI of the resulting lipid nanoparticles. The lipid nanoparticles according to Example 4 were tested by intra-tail vein administration to mice using similar materials and methods as in Example 1. The dosage of the lipid nanoparticles to mice was 200 μL (total amount of lipids: 5 mol/mouse).

### Comparative Example 4

### 4-2. Intravenous Administration of Lipid Nanoparticles (Phospholipid: DOPE, Average Particle Size: 30 nm) and Organ Isolation and Investigation on Intraorgan Distribution by Fluorescence Imaging Observation

In this Comparative Example, the lipid nanoparticles according to Comparative Example 4 were prepared and tested by intra-tail vein administration to mice using similar materials and methods as in Example 4 except that DOPE and CHEMS were used in place of DOPC which was used as phospholipid, and cholesterol in Example 4. Table 3 below shows the average particle size and PDI of the resulting lipid nanoparticles.

TABLE 3

| | Phospholipid | Sterol lipid | PEG-modified lipid | Average | |
|---|---|---|---|---|---|
| | (molar ratio) | (molar ratio) | (molar ratio) | particle size | PDI |
| Example 3 | DOPC | Cholesterol | DSG-PEG2000 | 121 nm | 0.278 |
| Example 4 | (60 mol) | (40 mol) | (5 mol) | 30 nm | 0.268 |
| Comparative | DOPE | cholesteryl | DSG-PEG2000 | 107 nm | 0.212 |
| Example 3 | (60 mol) | hemisuccinate | (5 mol) | 30 nm | 0.315 |
| Comparative | | (40 mol) | | | |
| Example 4 | | | | | |

### Results from Examples 3, 4 and Comparative Example 3, 4

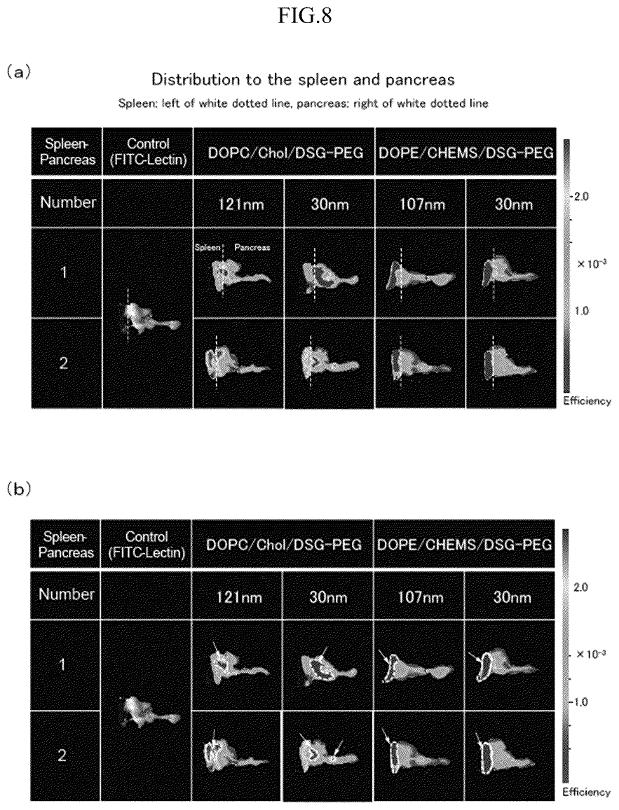

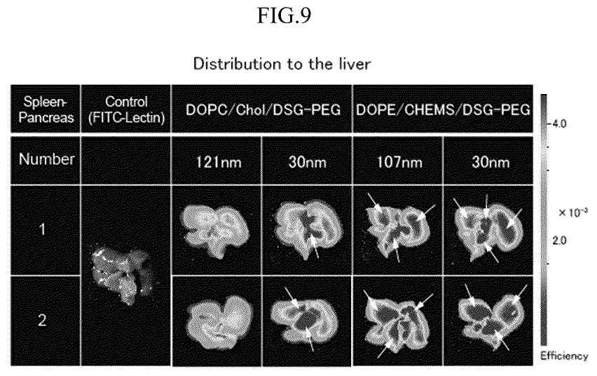

FIG. 8 shows results from ex vivo fluorescence imaging of pancreas and spleen removed 4 hours after the DiD-labeled lipid nanoparticles prepared in Examples 3, 4 and Comparative Examples 3, 4 were administered in tail vein to mice. White dotted lines in FIG. 8(a) indicate the boundaries between the spleen (left of the white dotted lines) and pancreas (right of the white dotted lines). In FIG. 8(b), the areas where the fluorescence intensity of DiD is more than 2.2×10⁻³, i.e., the high accumulation areas of lipid nanoparticles, are indicated by white dotted lines and white arrows. FIG. 9 shows results from ex vivo fluorescence imaging of liver removed 4 hours after the DiD-labeled lipid nanoparticles prepared in Examples 3, 4 and Comparative Examples 3, 4 were administered in tail vein to mice. With regard to white arrows in FIG. 9, the areas where the fluorescence intensity of DiD is more than 4.0×10⁻³ are indicated by the white arrows. These results reveal that the majority of the lipid nanoparticles comprising DOPE according to Comparative Examples 3, 4 accumulated in the liver and spleen whereas the lipid nanoparticles comprising DOPC according to Examples 3, 4 showed weak fluorescence in the liver and spleen and accumulated most in the pancreas. Comparison with the results from the lipid nanoparticles comprising DOPC (average particle size: 166 nm) according to Example 1 (see FIG. 1(b)) suggests that the smaller the particle sizes of the lipid nanoparticles are, the better the migration to the pancreas is.

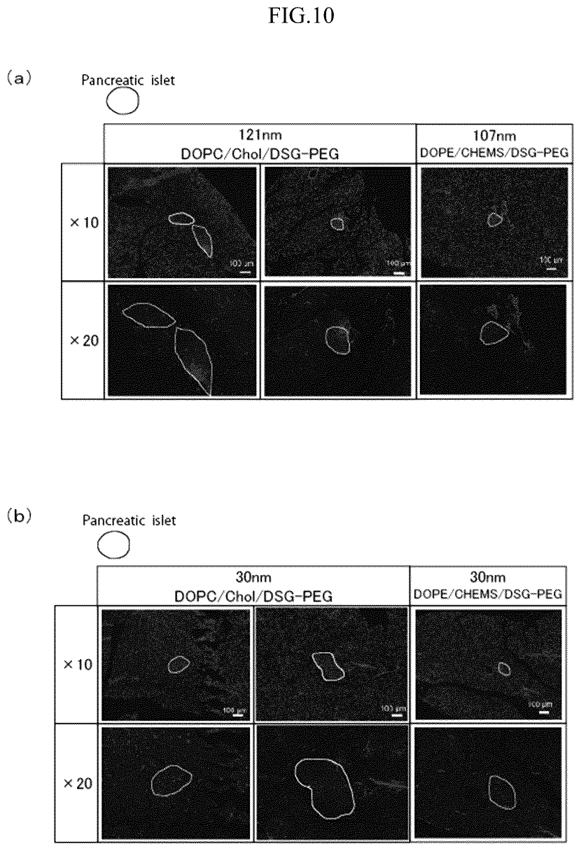

Next, FIG. 10 shows observation results from fluorescence microscopy on frozen sections prepared from pancreas removed 4 hours after the DiD-labeled lipid nanoparticles prepared in Examples 3, 4 and Comparative Examples 3, 4 were administered in tail vein to mice. White lines in each of the photographs indicate pancreatic islets. FIG. 10(a) shows results from Example 3 and Comparative Example 3, and FIG. 10(b) shows results from Example 4 and Comparative Example 4. The results reveal that the lipid nanoparticles comprising DOPE according to Comparative Examples 3, 4 did not show migration and accumulation to pancreatic islets whereas the lipid nanoparticles comprising DOPC according to Example 3 showed red fluorescence due to DiD within pancreatic islets surrounded by white lines, indicating migration and accumulation to pancreatic islets. In contrast, the results demonstrate that the small lipid nanoparticles with a particle size of 30 nm according to Example 4 did not show accumulation specific to pancreatic islets, but showed migration to exocrine tissues of the pancreas. This suggests that the small particles of 30 nm showed the most excellent migration to the pancreas, but they were so small that they did not stay in pancreatic islets and migrated smoothly to exocrine tissues in a short period of time. This reveals that the lipid nanoparticles prepared in Example 4 having a particle size of 30 nm showed excellent migration to exocrine tissues of the pancreas.

### [Mean Particle Sizes and Intraorgan Distributions of Lipid Nanoparticles]

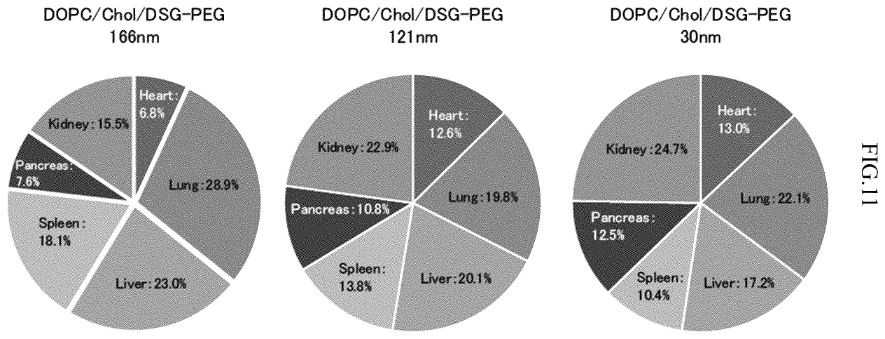

The ex vivo fluorescence imaging images of each of the organs removed 4 hours after the DiD-labeled lipid nanoparticles prepared in Examples 1, 3, and 4 were administered in tail vein to mice were ROI analyzed to quantify the fluorescence intensity of DiD in each of the organs, thereby calculating the fluorescence percentage (%) of each of the organs relative to the total fluorescence intensity (100%) of all of the organs. The results are shown in FIG. 11. The data for the fluorescence percentages (%) in the liver, spleen, and pancreas and the pancreatic migration are shown in Table 4 below. It is noted that the calculation was performed using the following formula: pancreatic migration=[fluorescence percentage of the pancreas/(fluorescence percentage of the liver+fluorescence percentage of the spleen)]×100.

TABLE 4

| | Example 1 | Example 3 | Example 4 |
|---|---|---|---|
| | DOPC/Chol/ | DOPC/Chol/ | DOPC/Chol/ |
| Components | DSG-PEG2000 | DSG-PEG2000 | DSG-PEG2000 |
| Average | 166 nm | 121 nm | 30 nm |
| particle size | | | |
| Liver (%) | 23.0 | 20.1 | 17.2 |
| Spleen (%) | 18.1 | 13.8 | 10.4 |
| Pancreas (%) | 7.6 | 10.8 | 12.5 |
| Pancreatic | 18.5 | 31.9 | 45.3 |
| migration (%) | | | |

These results indicate that all of the lipid nanoparticles showed high pancreatic migration of more than 18%, and the smaller the particle sizes of the lipid nanoparticles are, the better the migration to the pancreas is. These results also indicate that the lipid nanoparticles from these Examples all showed low migration to the liver and spleen, thus reducing the burden on these organs.

### Example 5

### 5-1. Intravenous Administration of Lipid Nanoparticles (Phospholipid: DOPC, Average Particle Size: 147 nm) and Organ Isolation and Investigation on Intraorgan Distribution by Fluorescence Imaging Observation

In this Example, the lipid nanoparticles according to Example 5 were prepared and tested by intra-tail vein administration to mice using similar materials and methods as in Example 1 described above. Table 5 below shows the average particle size and PDI of the resulting lipid nanoparticles. The dosage of the lipid nanoparticles to mice was 200 μL (total lipid content: 5 mol/mouse).

### Comparative Example 5

### 5-2. Intravenous Administration of Lipid Nanoparticles (Phospholipid: DSPC, Average Particle Size: 148 nm) and Organ Isolation and Investigation on Intraorgan Distribution by Fluorescence Imaging Observation

In this Comparative Example, the lipid nanoparticles according to Comparative Example 5 were prepared and tested by intra-tail vein administration to mice using similar materials and methods as in Comparative Example 1 described above. Table 5 below shows the average particle size and PDI of the resulting lipid nanoparticles. The dosage of the lipid nanoparticles to mice was 200 μL (total amount of lipid: 5 mol/mouse).

### Comparative Example 6

### 5-3. Intravenous Administration of Lipid Nanoparticles (Phospholipid: DOPE, Average Particle Size: 150 nm) and Organ Isolation and Investigation on Intraorgan Distribution by Fluorescence Imaging Observation

In this Comparative Example, the lipid nanoparticles according to Comparative Example 6 were prepared and tested by intra-tail vein administration to mice using similar materials and methods as in Comparative Example 5 except that DOPE and CHEMS were used in place of DOPC which was used as a phospholipid, and cholesterol in Comparative Example 5. Table 5 below shows the average particle size and PDI of the resulting lipid nanoparticles.

TABLE 5

| | Phospholipid | Sterol lipid | PEG-modified lipid | Average | |
|---|---|---|---|---|---|
| | (molar ratio) | (molar ratio) | (molar ratio) | particle size | PDI |
| Example 5 | DOPC | Cholesterol | DSG-PEG2000 | 147 nm | 0.248 |
| | (60 mol) | (40 mol) | (5 mol) | | |
| Comparative | DSPC | Cholesterol | DSG-PEG2000 | 148 nm | 0.340 |
| Example 5 | (60 mol) | (40 mol) | (5 mol) | | |
| Comparative | DOPE | cholesteryl | DSG-PEG2000 | 150 nm | 0.183 |
| Example 6 | (60 mol) | hemisuccinate | (5 mol) | | |
| | | (40 mol) | | | |

### Results from Examples 5 and Comparative Examples 5, 6

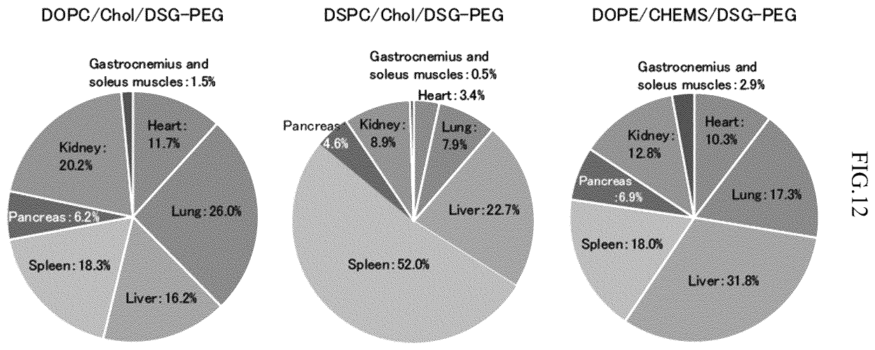

The ex vivo fluorescence imaging images of heart, lung, pancreas, spleen, liver, kidney, and gastrocnemius and soleus muscles removed 24 hours after the DiD-labeled lipid nanoparticles prepared in Example 5 and Comparative Examples 5, 6 were administered in tail vein to mice were captured. These images were ROI analyzed to quantify the fluorescence intensity of DiD in each organ. The data obtained for the fluorescence percentage (%) of each organ relative to the total fluorescence intensity of all organs (100%) are graphically shown in FIG. 12. The data for the fluorescence percentages (%) in the liver, spleen, and pancreas and the pancreatic migration are shown in Table 6 below. It is noted that the calculation was performed using the following formula: pancreatic migration=[fluorescence percentage of the pancreas/(fluorescence percentage of the liver+fluorescence percentage of the spleen)]×100.

TABLE 6

| | | Comparative | Comparative |
|---|---|---|---|
| | Example 5 | Example 5 | Example 6 |
| Components | DOPC/Chol/ | DSPC/Chol/ | DOPE/CHEMS/ |
| | DSG-PEG2000 | DSG-PEG2000 | DSG-PEG2000 |
| Average | 147 nm | 148 nm | 150 nm |
| particle size | | | |
| Liver (%) | 16.2 | 22.7 | 31.8 |
| Spleen (%) | 18.3 | 52.0 | 18.0 |
| Pancreas (%) | 6.2 | 4.6 | 6.9 |
| Pancreatic | 18.0 | 6.2 | 13.9 |
| migration (%) | | | |

These results reveal that the majority of the lipid nanoparticles according to Comparative Examples 5, 6 accumulated in the liver and spleen whereas the lipid nanoparticles comprising DOPC according to Example 5 showed less intensive distribution to the liver and spleen, indicating that pancreatic migration is high.

### Example 6

### 6. Investigation on Content Percentage of Cholesterol in Lipid Nanoparticles (Phospholipid: DOPC)

In this Example, lipid nanoparticles with various content percentages of a sterol lipid among the components constituting the lipid nanoparticles were prepared, and their intraorgan distributions were investigated by fluorescence imaging observation.

### 6-1. Intravenous Administration of Lipid Nanoparticles (0 Mol % Cholesterol) and Organ Isolation and Investigation on Intraorgan Distribution by Fluorescence Imaging Observation

In this Example, DOPC was used as a phospholipid, and DSG-PEG 2000 was used as a PEG-modified lipid, and no sterol lipid was included. The total lipid concentration of the sample solution to be pumped into the microfluidic device (final concentration 25 mM), and the total flow rate (TFR=250 mL/min) and the flow rate ratio (FRR=1) of the sample solution and the water-soluble solution were adjusted using a composition of DOPC:DSG-PEG 2000=100:5 (by molar ratio), thereby obtaining the lipid nanoparticles according to Example 6-1. The lipid nanoparticles according to Example 6-1 were prepared and tested by intra-tail vein administration to mice using similar materials and methods as in Example 1 except for these. The dosage of the lipid nanoparticles to mice was 200 μL of a dispersion of the prepared DiD-labeled lipid nanoparticles (total amount of lipids: 5 mol/mouse). Table 7 below shows the average particle size and PDI of the resulting lipid nanoparticles.

### 6-2. Intravenous Administration of Lipid Nanoparticles (19 Mol % Cholesterol) and Organ Isolation and Investigation on Intraorgan Distribution by Fluorescence Imaging Observation

In this Example, DOPC was used as a phospholipid, and cholesterol (Chol) was used as a sterol lipid, and DSG-PEG 2000 was used as a PEG-modified lipid with a composition of DOPC:Chol:DSG-PEG 2000=80:20:5 (by molar ratio), and the total lipid concentration of the sample solution to be pumped into the microfluidic device (final concentration 25 mM), and the total flow rate (TFR=250 mL/min) and the flow rate ratio (FRR=1) of the sample solution and the water-soluble solution were adjusted, thereby obtaining the lipid nanoparticles according to Example 6-2. The lipid nanoparticles according to Example 6-2 were prepared and tested by intra-tail vein administration to mice using similar materials and methods as in Example 1 except for these. The dosage of the lipid nanoparticles to mice was 200 μL of a dispersion of the prepared DiD-labeled lipid nanoparticles (total amount of lipids: 5 μmol/mouse). Table 7 below shows the average particle size and PDI of the resulting lipid nanoparticles.

### 6-3. Intravenous Administration of Lipid Nanoparticles (38 Mol % Cholesterol) and Organ Isolation and Investigation on Intraorgan Distribution by Fluorescence Imaging Observation

In this Example, DOPC was used as a phospholipid, and cholesterol (Chol) was used as a sterol lipid, and DSG-PEG 2000 was used as a PEG-modified lipid with a composition of DOPC:Chol:DSG-PEG 2000=60:40:5 (by molar ratio), and the total lipid concentration of the sample solution to be pumped into the microfluidic device (final concentration 25 mM), and the total flow rate (TFR=250 mL/min) and the flow rate ratio (FRR=1) of the sample solution and the water-soluble solution were adjusted, thereby obtaining the lipid nanoparticles according to Example 6-3. The lipid nanoparticles according to Example 6-3 were prepared and tested by intra-tail vein administration to mice using similar materials and methods as in Example 1 except for these. The dosage of the lipid nanoparticles to mice was 200 μL of a dispersion of the prepared DiD-labeled lipid nanoparticles (total amount of lipids: 5 μmol/mouse). Table 7 below shows the average particle size and PDI of the resulting lipid nanoparticles.

### 6-4. Intravenous Administration of Lipid Nanoparticles (57 Mol % Cholesterol) and Organ Isolation and Investigation on Intraorgan Distribution by Fluorescence Imaging Observation

In this Example, DOPC was used as a phospholipid, and cholesterol (Chol) was used as a sterol lipid, and DSG-PEG 2000 was used as a PEG-modified lipid with a composition of DOPC:Chol:DSG-PEG 2000=40:60:5 (by molar ratio), and the total lipid concentration of the sample solution to be pumped into the microfluidic device (final concentration 25 mM), and the total flow rate (TFR=250 mL/min) and the flow rate ratio (FRR=1.5) of the sample solution and the water-soluble solution were adjusted, thereby obtaining the lipid nanoparticles according to Example 6-4. The lipid nanoparticles according to Example 6-4 were prepared and tested by intra-tail vein administration to mice using similar materials and methods as in Example 1 except for these. The dosage of the lipid nanoparticles to mice was 200 μL of a dispersion of the prepared DiD-labeled lipid nanoparticles (total lipid content: 5 mol/mouse). Table 7 below shows the average particle size and PDI of the resulting lipid nanoparticles.

TABLE 7

| | | | PEG-modified | Average | |
|---|---|---|---|---|---|
| | Phospholipid | Sterol lipid | lipid | particle | |
| | (molar ratio) | (molar ratio) | (molar ratio) | size | PDI |
| Example | DOPC | None | DSG-PEG2000 | 153 nm | 0.143 |
| 6-1 | (100 mol) | (0 mol) | (5 mol) | | |
| Example | DOPC | Cholesterol | DSG-PEG2000 | 164 nm | 0.138 |
| 6-2 | (80 mol) | (20 mol) | (5 mol) | | |
| Example | DOPC | Cholesterol | DSG-PEG2000 | 133 nm | 0.094 |
| 6-3 | (60 mol) | (40 mol) | (5 mol) | | |
| Example | DOPC | Cholesterol | DSG-PEG2000 | 94 nm | 0.196 |
| 6-4 | (40 mol) | (60 mol) | (5 mol) | | |

### Results from Example 6

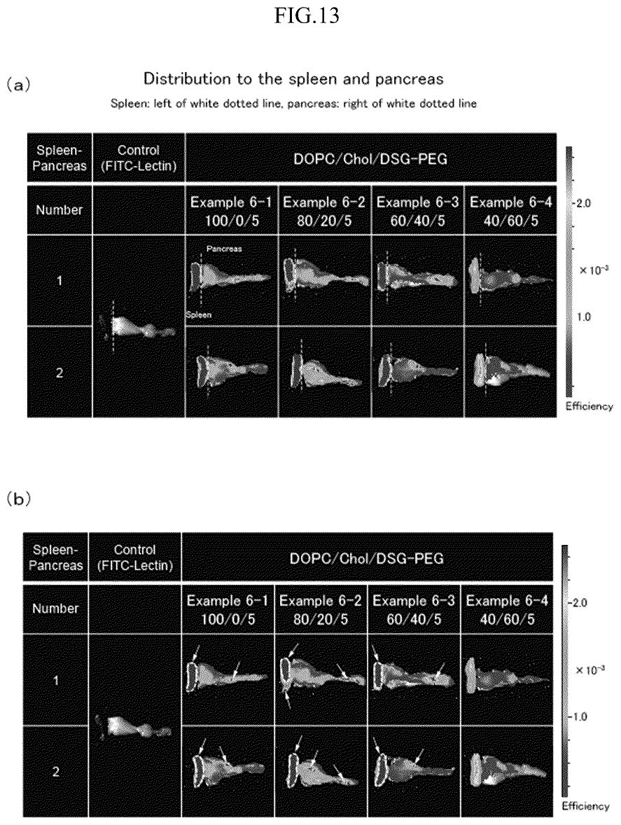

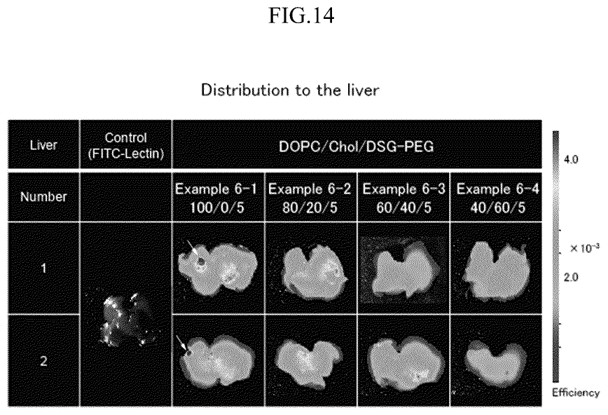

FIG. 13 shows results from ex vivo fluorescence imaging of pancreas and spleen removed 4 hours after the DiD-labeled lipid nanoparticles each prepared in Example 6 were administered in tail vein to mice. White dotted lines in FIG. 13(a) indicate the boundaries between the spleen (left of the white dotted lines) and pancreas (right of the white dotted lines). In FIG. 13(b), the areas where the fluorescence intensity of DiD is more than 2.2×10⁻³, i.e., the high accumulation areas of lipid nanoparticles, are indicated by white dotted lines and white arrows. FIG. 14 shows results from ex vivo fluorescence imaging of liver removed 4 hours after the DiD-labeled lipid nanoparticles each prepared in Example 6 were administered in tail vein to mice. With regard to white arrows in FIG. 14, the areas where the fluorescence intensity of DiD is more than 4.0×10⁻³ are indicated by the white arrows. These results indicate that no significant difference was observed among the lipid nanoparticles of Example 6-1 including no cholesterol at all, and the lipid nanoparticles of Example 6-2 (comprising 19 mol % cholesterol) and Example 6-3 (comprising 38 mol % cholesterol). In contrast, the lipid nanoparticles according to Example 6-4, which include the highest percentage of cholesterol (comprising 57 mol % cholesterol), showed weak fluorescence in the spleen and liver, suggesting that the high cholesterol content constituting the lipid nanoparticles may have reduced their uptake into liver Kupffer cells.

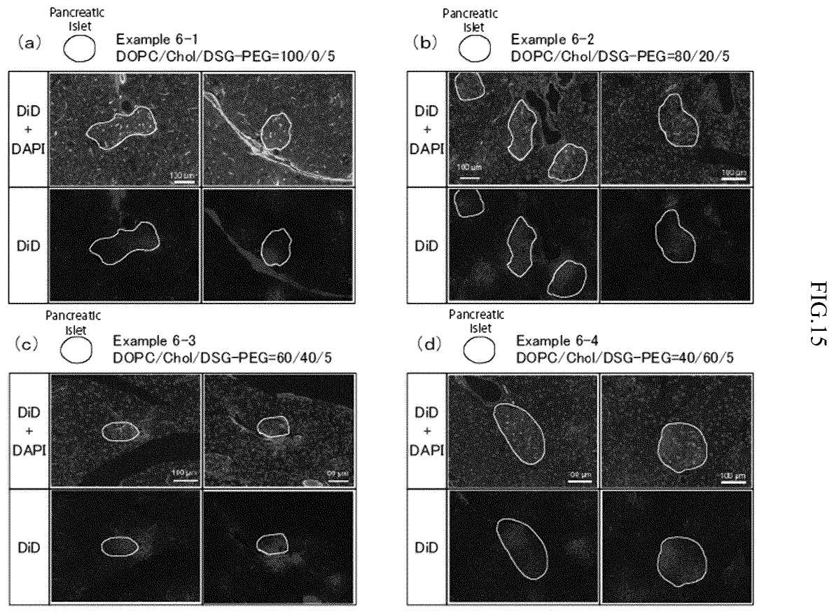

FIG. 15 shows observation results from fluorescence microscopy on frozen sections prepared from pancreas removed 4 hours after the lipid nanoparticles according to Example 6 were each administered in tail vein to mice. White lines in the photographs indicate pancreatic islets. FIG. 15(a) shows results from Example 6-1, and FIG. 15(b) shows results from Example 6-2, and FIG. 15(c) shows results from Example 6-3, and FIG. 15(d) shows results from Example 6-4. These results demonstrate that all of the lipid nanoparticles according to Example 6 migrated and accumulated to pancreatic islets. This indicates that migration and accumulation to pancreatic islets can occur without cholesterol among the lipid components constituting the lipid nanoparticles, and that DOPC as a phospholipid can play an important role in migration and accumulation to pancreatic islets (pancreas). The results in FIG. 15 also demonstrate that the lipid nanoparticles according to Example 6-3 (comprising 38 mol % cholesterol) and Example 6-4 (comprising 57 mol % cholesterol) showed significant migration and accumulation especially to pancreatic islets.

### Example 7

### 7. Preparation of Lipid Nanoparticles Containing a Drug for Treating a Pancreatic Disease

In this Example, nobiletin (hereafter also referred to as NOB), a methylated flavonoid contained in citrus fruit skin, as an example of a drug for treating diabetes which targets pancreatic islets, was contained in the lipid nanoparticles (components: DOPC/Chol/DSG-PEG) investigated up to Example 6, thereby preparing lipid nanoparticles containing a drug for treating a pancreatic disease.

### 7-1. Preparation of Nobiletin (NOB)-Containing Lipid Nanoparticles (1)

First, nobiletin powder (Ushio ChemiX Corporation) was dissolved in ethanol to prepare a stock solution. In this Example, a sample solution (final lipid concentration: 2.5 μM) to be pumped into the microfluidic device was prepared in which DOPC was used as a phospholipid, cholesterol (Chol) was used as a sterol lipid, and DSG-PEG 2000 was used as a PEG-modified lipid with a composition of DOPC:Chol:DSG-PEG2000:NOB=60:40:5:10 (by molar ratio). The total flow rate (TFR=150 mL/min) and the flow rate ratio (FRR=1) of the sample solution and the water-soluble solution were adjusted, and the lipid nanoparticles according to Example 7-1 were prepared using similar materials and methods as in Example 1 except for these. Table 8 below shows the average particle size, PDI, and the enclosure percentage of nobiletin of the resulting lipid nanoparticles. It is noted that the enclosure percentage of nobiletin was measured as follows.

### [Measurement of the Enclosure Percentage of Nobiletin]

A dispersion containing the prepared lipid nanoparticles was dialyzed against a PBS solution (pH 7.4) for 2 hours or more, and then Triton® X-100 as a nonionic surfactant was added to give a final concentration of 0.2 w/v %, and mixed by vortex. After mixing, the dispersion was subjected to ultrasonic waves for 15 minutes in a bath-type ultrasonic device, and then centrifuged at 14,000 rpm for 20 minutes at 4° C. to completely disrupt the lipid nanoparticles in the dispersion. After confirming that there was no post-centrifugation precipitation in the solution, the solution was filtered through a filter with a pore diameter of 200 nm. The concentration of nobiletin in the solution was then measured by high-performance liquid chromatography (HPLC). HPLC measurement conditions are shown below.

### HPLC Measurement Conditions

Detection: UV-vis, 254 nm
Column: Develosil ODS-HG (5 m), 4.6 mm φ×150 mm
Column temperature: 35° C.
Mobile phase: MeOH:H₂0=72:28
Flow rate: 1.0 mL/h
Injection volume: 10 μL

Based on the concentration of nobiletin measured, the enclosure percentage was calculated by the following formula:

$$\\ Enclosure percentage \% = ( amount of nobiletin in solution after dialysis / theoretical content ) \times 100$$

### 7-2. Preparation of NOB-Containing Lipid Nanoparticles (2)

In this Example, the lipid nanoparticles according to Example 7-2 were prepared using similar materials and methods as in Example 7-1 described above except that the total flow rate and the flow rate ratio of the sample solution and the water-soluble solution to be pumped into the microfluidic device in Example 7-1 were changed to TFR=1000 mL/min and FRR=3, respectively. Table 8 below shows the average particle size, PDI, and the enclosure percentage of nobiletin of the resulting lipid nanoparticles.

### 7-3. Preparation of NOB-Containing Lipid Nanoparticles (3)

In this Example, the lipid nanoparticles according to Example 7-3 were prepared using similar materials and methods as in Example 7-1 described above except that a sample solution (final lipid concentration: 2.5 μM) to be pumped into the microfluidic device was prepared in which DOPC was used as a phospholipid, cholesterol (Chol) was used as a sterol lipid, and DSG-PEG 2000 was used as a PEG-modified lipid with a composition of DOPC:Chol:DSG-PEG 2000:NOB=50:40:5:10 (by molar ratio). Table 8 below shows the average particle size, PDI, and the enclosure percentage of nobiletin of the resulting lipid nanoparticles.

### 7-4. Preparation of NOB-Containing Lipid Nanoparticles (4)

In this Example, the lipid nanoparticles according to Example 7-4 were prepared using similar materials and methods as in Example 7-1 described above except that a sample solution (final lipid concentration: 2.5 μM) to be pumped into the microfluidic device was prepared in which DOPC was used as a phospholipid, cholesterol (Chol) was used as a sterol lipid, and DSG-PEG 2000 was used as a PEG-modified lipid with a composition of DOPC:Chol:DSG-PEG 2000:NOB=60:40:5:5 (by molar ratio). Table 8 below shows the average particle size, PDI, and the enclosure percentage of nobiletin of the resulting lipid nanoparticles.

### 7-5. Preparation of NOB-Containing Lipid Nanoparticles (5)

In this Example, the lipid nanoparticles according to Example 7-5 were prepared using similar materials and methods as in Example 7-4 described above except that the total flow rate and the flow rate ratio of the sample solution and the water-soluble solution to be pumped into the microfluidic device in Example 7-4 were changed to TFR=1000 mL/min and FRR=3, respectively. Table 8 below shows the average particle size, PDI, and the enclosure percentage of nobiletin of the resulting lipid nanoparticles.

### 7-6. Preparation of NOB-Containing Lipid Nanoparticles (6)

In this Example, the lipid nanoparticles according to Example 7-6 were prepared using similar materials and methods as in Example 7-1 described above except that a sample solution (final lipid concentration: 2.5 μM) to be pumped into the microfluidic device was prepared in which DOPC was used as a phospholipid, cholesterol (Chol) was used as a sterol lipid, and DSG-PEG 2000 was used as a PEG-modified lipid with a composition of DOPC:Chol:DSG-PEG 2000:NOB=50:40:5:5 (by molar ratio). Table 8 below shows the average particle size, PDI, and the enclosure percentage of nobiletin of the resulting lipid nanoparticles.

TABLE 8

| | Lipid | | | | Average | | Enclosure |
|---|---|---|---|---|---|---|---|
| | Phospholipid | Sterol lipid | PEG-modified lipid | | particle size | | percentage |
| Composition | (molar ratio) | (molar ratio) | (molar ratio) | Drug | (nm) | PDI | (%) |
| Example 7-1 | DOPC | Cholesterol | DSG-PEG2000 | Nobiletin | 148 | 0.223 | 50.8 |
| | (60 mol) | (40 mol) | (5 mol) | (10 mol) | | | |
| Example 7-2 | DOPC | Cholesterol | DSG-PEG2000 | Nobiletin | 33 | 0.257 | 57.8 |
| | (60 mol) | (40 mol) | (5 mol) | (10 mol) | | | |
| Example 7-3 | DOPC | Cholesterol | DSG-PEG2000 | Nobiletin | 143 | 0.229 | 52.5 |
| | (50 mol) | (40 mol) | (5 mol) | (10 mol) | | | |
| Example 7-4 | DOPC | Cholesterol | DSG-PEG2000 | Nobiletin | 153 | 0.215 | 49.7 |
| | (60 mol) | (40 mol) | (5 mol) | (5 mol) | | | |
| Example 7-5 | DOPC | Cholesterol | DSG-PEG2000 | Nobiletin | 29 | 0.223 | 57.9 |
| | (60 mol) | (40 mol) | (5 mol) | (5 mol) | | | |
| Example 7-6 | DOPC | Cholesterol | DSG-PEG2000 | Nobiletin | 146 | 0.240 | 48.7 |
| | (50 mol) | (40 mol) | (5 mol) | (5 mol) | | | |

### Example 8

### 8. Preparation of Lipid Nanoparticles Containing Nucleic Acid

In this Example, nucleic acid-containing lipid nanoparticles containing siRNA (MALAT-1 from GeneDesign, Inc.) were prepared as an example of nucleic acid medicament.

### 8-1. Preparation of Nucleic Acid-Containing Lipid Nanoparticles (1)

In this Example, in order to internalize siRNA into lipid nanoparticles, D-Lin-MC3-DMA (MC3) was used as an ionized lipid which is positively charged at pH (4.0) during preparation and can electrostatically interact with negatively charged siRNA, in addition to DOPC as a phospholipid, cholesterol (Chol) as a sterol lipid, and DMG-PEG 2000 as a PEG-modified lipid. A sample solution (final lipid concentration: 2.5 μM) to be pumped into the microfluidic device was prepared using a composition of MC3:DOPC:Chol:DMG-PEG 2000=50:20:35:5 (by molar ratio). siRNA (MALAT-1 from GeneDesign, Inc.) was mixed with 25 mM acetate buffer (pH 4.0) as a water-soluble solution so that the blending ratio of the lipid components and the water-soluble solution was MC3:DOPC:Chol:DMG-PEG 2000:siRNA=50:20:35:5:0.2 by molar ratio. It is noted that the N/P ratio was 6. For the purpose of increasing particle sizes, NaCl was also added to this water-soluble solution to give a concentration of 300 mM. The lipid nanoparticle-containing dispersion according to Example 8-1 were obtained using similar materials and methods as in Example 1 except that the total flow rate (TFR=250 mL/min) and the flow rate ratio (FRR=3) of the sample solution and the water-soluble solution were adjusted. The resulting lipid nanoparticle-containing dispersion was dialyzed against MES (20 mM, pH 5.5) for 2 hours, and then against HEPES (10 mM, pH 7.4) overnight. Table 9 below shows the average particle size, PDI, and the enclosure percentage of siRNA of the resulting lipid nanoparticles. It is noted that the enclosure percentage of siRNA was measured as follows.

[Measurement of Enclosure Percentage of siRNA]

First, 8 siRNA solutions having siRNA concentrations ranging from 0 to 200 μg/100 μL were prepared as standard liquids for obtaining a calibration curve, and 100 μL of each was placed in a 96-well plate. A solution containing the lipid nanoparticles prepared was then diluted with nuclease-free water to 0.1 μg/100 μL which is a theoretical value at 100% siRNA encapsulation, and 100 μL of each was placed in the 96-well plate. A 100 μl solution of RiboGreen diluted 200 fold with 1×TE buffer was added to each well, and allowed to stand at room temperature for 2 to 5 minutes. A microplate reader was used for measurements at an excitation wavelength of 485 nm and a fluorescence wavelength of 535 nm to obtain a “Ffree” value. After 20 μL of 2% Triton® X-100 solution was added to each well of the same plate to disrupt the lipid nanoparticles, and measurements were performed in a similar manner with the microplate reader to obtain a “Ftotal” value. Based on the measured values and the following formula, the enclosure percentages of siRNA of the lipid nanoparticles were calculated.

Enclosure percentage %=(Ffree−Ftotal)/Ftotal×100

### 8-2. Preparation of Nucleic Acid-Containing Lipid Nanoparticles (2)

In this Example, the lipid nanoparticles according to Example 8-2 were prepared using similar materials and methods as in Example 8-1 described above except that NaCl was not added to the water-soluble solution to be pumped into the microfluidic device in Example 8-1 (NaCl concentration: 0 mM), and the total flow rate of the sample solution and the water-soluble solution to be pumped into the microfluidic device was changed to TFR=2000 mL/min. Table 9 below shows the average particle size, PDI, and the enclosure percentage of siRNA of the resulting lipid nanoparticles.

### 8-3. Preparation of Nucleic Acid-Containing Lipid Nanoparticles (3)

In this Example, siRNA was mixed with the water-soluble solution to be pumped into the microfluidic device in Example 8-2 so that the blending ratio of the lipid components and siRNA was MC3:DOPC:Chol:DMG-PEG 2000:siRNA=50:20:35:5:0.4 by molar ratio. It is noted that the N/P ratio was 3 in this case. The lipid nanoparticles according to Example 8-3 were prepared using similar materials and methods as in Example 8-2 described above except for these. Table 9 below shows the average particle size, PDI, and the enclosure percentage of siRNA of the resulting lipid nanoparticles.

TABLE 9

| | Lipid | | | | | | Average | | Enclosure |
|---|---|---|---|---|---|---|---|---|---|
| | Ionized Lipid | Phospholipid | Stero1 lipid | PEG-modified lipid | Nucleic | N/P | particle | | percentage |
| Composition | (molar ratio) | (molar ratio) | (molar ratio) | (molar ratio) | acid | ratio | size (nm) | PDI | (%) |
| Example 8-1 | MC3 | DOPC | Cholesterol | DMG-PEG2000 | siRNA | 6 | 144 | 0.315 | 83.6 |
| | (50 mol) | (20 mol) | (35 mol) | (5 mol) | (0.2 mol) | | | | |
| Example 8-2 | MC3 | DOPC | Cholesterol | DMG-PEG2000 | siRNA | 6 | 40 | 0.082 | 92.6 |
| | (50 mol) | (20 mol) | (35 mol) | (5 mol) | (0.2 mol) | | | | |
| Example 8-3 | MC3 | DOPC | Cholesterol | DMG-PEG2000 | siRNA | 3 | 40 | 0.071 | 88.0 |
| | (50 mol) | (20 mol) | (35 mol) | (5 mol) | (0.4 mol) | | | | |

### Example 9

### 9. Investigation on the Content Percentage of a PEG-Modified Lipid in Lipid Nanoparticles (Phospholipid: DOPC)

In this Example, lipid nanoparticles with various blending percentages of a PEG-modified lipid among the components constituting the lipid nanoparticles were prepared, and their intraorgan distributions were investigated by fluorescence imaging observation.

### Example 9-1

### 9-1. Intravenous Administration of Lipid Nanoparticles (1.5 Mol % PEG-Modified Lipid) and Organ Isolation and Investigation on Intraorgan Distribution by Fluorescence Imaging Observation

In this Example, DOPC was used as a phospholipid, and cholesterol (Chol) was used as a sterol lipid, and DSG-PEG 2000 was used as a PEG-modified lipid with a composition of DOPC:Chol:DSG-PEG 2000=60:40:1.5 (by molar ratio), and the total lipid concentration of the sample solution to be pumped into the microfluidic device (final concentration 25 mM), and the total flow rate (TFR=250 mL/min) and the flow rate ratio (FRR=1) of the sample solution and the water-soluble solution were adjusted, thereby obtaining the lipid nanoparticles according to Example 9-1. It is noted that the water-soluble solution was a PBS solution to which NaCl was added to give a concentration of 250 mM. The lipid nanoparticles according to Example 9-1 were prepared using similar materials and methods as in Example 1 except for these. Table 10 below shows the average particle size and PDI of the resulting lipid nanoparticles.

### Example 9-2

### 9-2. Intravenous Administration of Lipid Nanoparticles (2.9 Mol % PEG-Modified Lipid) and Organ Isolation and Investigation on Intraorgan Distribution by Fluorescence Imaging Observation

In this Example, the lipid nanoparticles according to Example 9-2 were obtained using similar materials and materials as Example 9-1 described above except that DOPC was used as a phospholipid, and cholesterol (Chol) was used as a sterol lipid, and DSG-PEG 2000 was used as a PEG-modified lipid with a composition of DOPC:Chol:DSG-PEG 2000=60:40:3 (by molar ratio), and the total lipid concentration of the sample solution to be pumped into the microfluidic device (final concentration 25 mM), and the total flow rate (TFR=250 mL/min) and the flow rate ratio (FRR=1) of the sample solution and the water-soluble solution were adjusted. Table 10 below shows the average particle size and PDI of the resulting lipid nanoparticles.

### Example 9-3

### 9-3. Intravenous Administration of Lipid Nanoparticles (4.8 Mol % PEG-Modified Lipid) and Organ Isolation and Investigation on Intraorgan Distribution by Fluorescence Imaging Observation

In this Example, the lipid nanoparticles according to Example 9-3 were obtained using similar materials and materials as Example 9-1 described above except that DOPC was used as a phospholipid, and cholesterol (Chol) was used as a sterol lipid, and DSG-PEG 2000 was used as a PEG-modified lipid with a composition of DOPC:Chol:DSG-PEG 2000=60:40:5 (by molar ratio), and the total lipid concentration of the sample solution to be pumped into the microfluidic device (final concentration 25 mM), and the total flow rate (TFR=250 mL/min) and the flow rate ratio (FRR=1) of the sample solution and the water-soluble solution were adjusted. Table 10 below shows the average particle size and PDI of the resulting lipid nanoparticles.

### Comparative Example 7

### 9-4. Intravenous Administration of Lipid Nanoparticles (0 Mol % PEG-Modified Lipid) and Organ Isolation and Investigation on Intraorgan Distribution by Fluorescence Imaging Observation

In this Comparative Example, DOPC was used as a phospholipid, cholesterol (Chol) was used as a sterol lipid, but a PEG-modified lipid was not included. The lipid nanoparticles according to Comparative Example 7 were obtained using similar materials and materials as Example 9-1 described above except that the total lipid concentration of the sample solution to be pumped into the microfluidic device (final concentration 25 mM), and the total flow rate (TFR=250 mL/min) and the flow rate ratio (FRR=3) of the sample solution and the water-soluble solution were adjusted with a composition of DOPC:Chol=60:40 (by molar ratio). Table 10 below shows the average particle size and PDI of the resulting lipid nanoparticles.

TABLE 10

| | Phospho- | | | | |
|---|---|---|---|---|---|
| | lipid | | PEG-modified | Average | |
| | (molar | Sterol lipid | lipid | particle | |
| | ratio) | (molar ratio) | (molar ratio) | size | PDI |
| Comparative | DOPC | Cholesterol | None | 100 nm | 0.145 |
| Example 7 | (60 mol) | (40 mol) | (0 mol) | | |
| Example 9-1 | DOPC | Cholesterol | DSG-PEG2000 | 132 nm | 0.230 |
| | (60 mol) | (40 mol) | (1.5 mol) | | |
| Example 9-2 | DOPC | Cholesterol | DSG-PEG2000 | 146 nm | 0.273 |
| | (60 mol) | (40 mol) | (3 mol) | | |
| Example 9-3 | DOPC | Cholesterol | DSG-PEG2000 | 148 nm | 0.232 |
| | (60 mol) | (40 mol) | (5 mol) | | |

For administration of lipid nanoparticles, 10-week old male C57BL/6J mice (Japan SLC, Inc.) were used. These C57BL/6J mice were administered in tail vein with 200 μL (total amount of lipids: 5 mol/mouse) of each of the DiD-labeled lipid nanoparticle dispersions prepared in this Example and Comparative Example. After 24 hours of administration, DyLight 488-labeled lectin (Lycopersicon esculentum (Tomato) Lectin-LEA, DyLight 488 from Thermo Fisher Scientific, Inc.) as a vascular labeling reagent was diluted 4 fold with PBS, and 200 μL (50 g in terms of LEL) was administered in tail vein. Each of the mice was anesthetized by inhalation of isoflurane 5 minutes after administration of DyLight488-labeled lectin, and blood was drawn from the inferior vena cava, and the heart, lung, pancreas, spleen, liver, kidney, and gastrocnemius and soleus muscles were isolated.

All of the isolated organs were observed using a fluorescence imaging system (IVIS Lumina III, Summit Pharmaceuticals International Corporation) at Ex/Em: 644 nm/663 nm, and the resulting fluorescence imaging images were ROI analyzed to quantify the fluorescence intensity of DiD (indicating the distribution of lipid nanoparticles) in each organ.

Frozen specimens of the isolated pancreas were also prepared. The isolated pancreas was immersed into and fixed with a solution of 4% paraformaldehyde and then impregnated with 10% sucrose for 4 hours, followed by 15% sucrose for 4 hours. It was then impregnated with 20% sucrose and allowed to stand overnight. Into a Cryomold, added and mixed were 300 μL of PBS and an O.C.T. Compound, to which the pancreas was then embedded. It was then frozen in liquid nitrogen and stored at −80° C. The frozen pancreatic specimen prepared was thinly sliced at a thickness of 5 m in a cryostat, which was then attached to a poly-L-lysine-coated glass slide. This was sealed with a nuclear staining sealant (DAPI-Fluoromount-G® from Cosmo Bio Co., Ltd.). The slide was observed and analyzed with a fluorescence microscope (BZ-X810 from Keyence Corporation).

### Results from Example 9 and Comparative Example 7

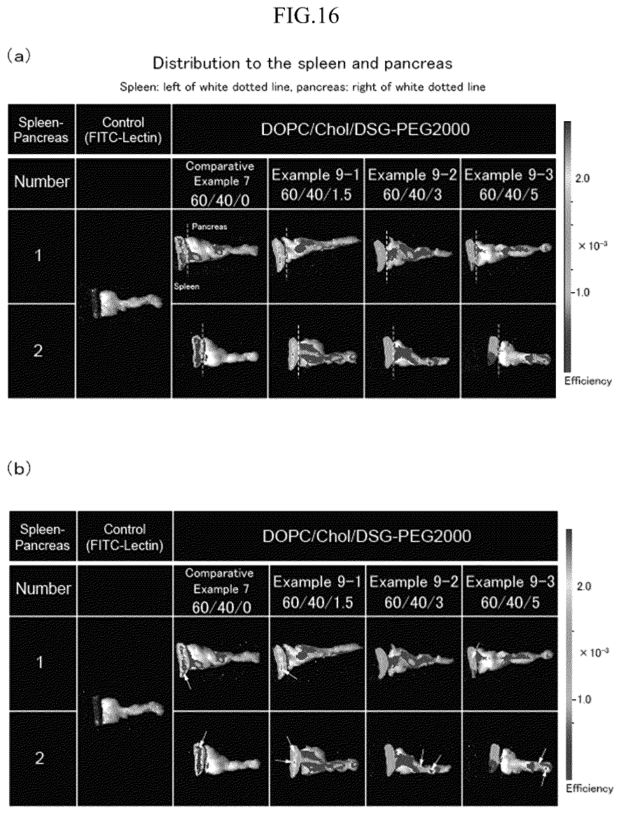

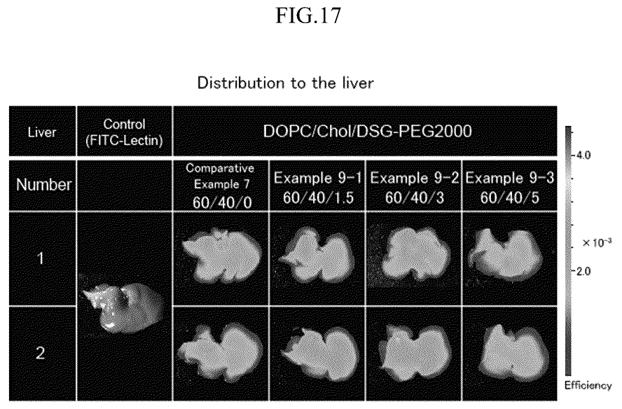

FIG. 16 shows results from ex vivo fluorescence imaging of pancreas and spleen removed 24 hours after the DiD-labeled lipid nanoparticles each prepared in Example 9 and Comparative Example 7 were administered in tail vein to mice. White dotted lines in FIG. 16(a) indicate the boundaries between the spleen (left of the white dotted lines) and pancreas (right of the white dotted lines). In FIG. 16(b), the high accumulation areas of lipid nanoparticles where the fluorescence intensity of DiD is more than 2.2×10⁻³ are indicated by white dotted lines and white arrows. Further, FIG. 17 shows results from ex vivo fluorescence imaging of liver removed 24 hours after the DiD-labeled lipid nanoparticles each prepared in Example 9 and Comparative Example 7 were administered in tail vein to mice. In FIG. 17, no areas where the fluorescence intensity of DiD was more than 4.0×10⁻³ were observed. These results revel that the lipid nanoparticles comprising no PEG-modified lipid according to Comparative Example 7 accumulated and remained in the spleen even after 24 hours of administration whereas the lipid nanoparticles comprising a PEG-modified lipid according to Example 9 showed weak fluorescence in the spleen, and migrated and accumulated in the pancreas.

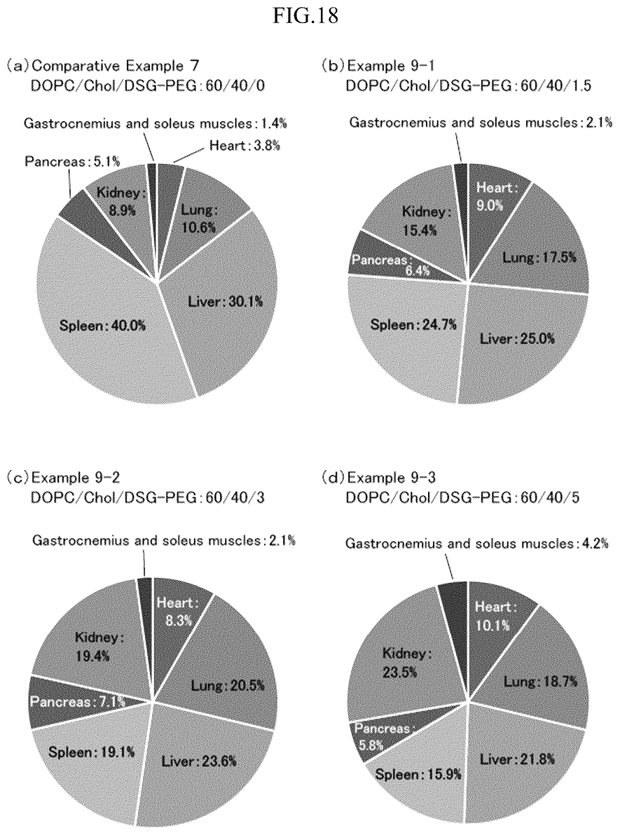

The ex vivo fluorescence imaging images of heart, lung, pancreas, spleen, liver, kidney, and gastrocnemius and soleus muscles removed 24 hours after the DiD-labeled lipid nanoparticles each prepared in Example 9 and Comparative Example 7 were administered in tail vein to mice were captured. These images were ROI analyzed to quantify the fluorescence intensity of DiD in each organ. The data obtained for the fluorescence percentage (%) of each organ relative to the total fluorescence intensity of all organs (100%) are shown in a pie graph in FIG. 18. The data for the percentages (%) of fluorescence in the liver, spleen, and pancreas, and pancreatic migration are shown in Table 11 below. It is noted that the calculation was performed using the following formula: pancreatic migration=[fluorescence percentage of the pancreas/(fluorescence percentage of the liver+fluorescence percentage of the spleen)]×100.

TABLE 11

| | Comparative | | | |
|---|---|---|---|---|
| | Example 7 | Example 9-1 | Example 9-2 | Example 9-3 |
| Components | DOPC/Chol/ | DOPC/Chol/ | DOPC/Chol/ | DOPC/Chol/ |
| | DSG-PEG2000 | DSG-PEG2000 | DSG-PEG2000 | DSG-PEG2000 |
| Component | 60/40/0 | 60/40/1.5 | 60/40/3 | 60/40/5 |
| molar ratio | | | | |
| Liver (%) | 30.1 | 25.0 | 23.6 | 21.8 |
| Spleen (%) | 40.0 | 24.7 | 19.1 | 15.9 |
| Pancreas (%) | 5.1 | 6.4 | 7.1 | 5.8 |
| Pancreatic migration | 7.3 | 12.9 | 16.6 | 15.4 |
| (%) | | | | |

These results reveal that the majority of the lipid nanoparticles according to Comparative Example 7 accumulated in the liver and spleen whereas the lipid nanoparticles comprising a PEG-modified lipid according to Examples 9-1 to 9-3 showed reduced distribution in the liver and spleen, indicating that pancreatic migration is high. Comparison of the results from Examples 9-1, 9-2, and 9-3 demonstrates that higher pancreatic migration was observed for the lipid nanoparticles according to Example 9-2 (comprising 2.9 mol % PEG-modified lipid) and Example 9-3 (comprising 4.8 mol % PEG-modified lipid).

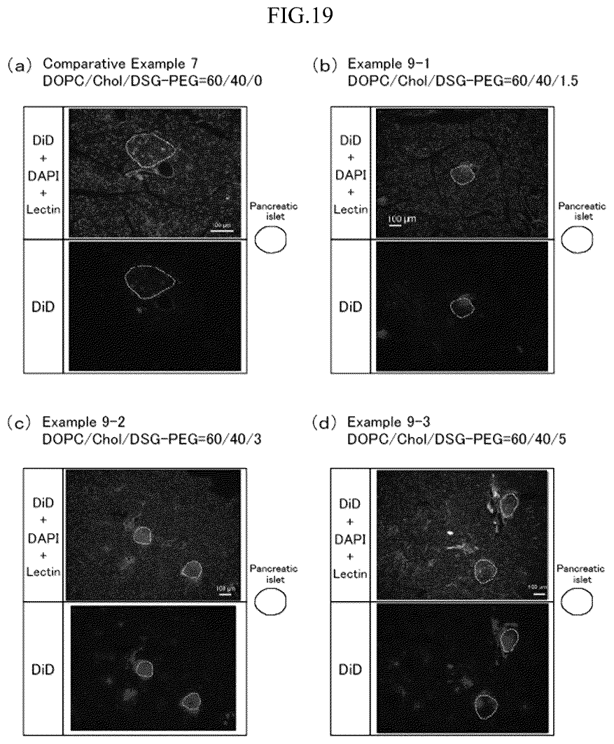

FIG. 19 shows observation results from fluorescence microscopy on frozen sections prepared from pancreas removed 24 hours after the lipid nanoparticles according to Example 9 and Comparative Example 7 were each administered in tail vein to mice. White lines in the photographs indicate pancreatic islets. FIG. 19(a) shows results from Comparative Example 7, and FIG. 19(b) shows results from Example 9-1, and FIG. 19(c) shows results from Example 9-2, and FIG. 19(d) shows results from Example 9-3. These results demonstrate that all of the lipid nanoparticles according to Example 9 showed migration and accumulation in pancreatic islets, and that the higher content of the PEG-modified lipid constituting the lipid nanoparticles lead to more significant migration and accumulation in pancreatic islets. However, the lipid nanoparticles according to Example 9-2 (comprising 2.9 mol % PEG-modified lipid) and the lipid nanoparticles according to Example 9-3 (comprising 4.8 mol % PEG-modified lipid) showed no significant difference in their distribution to pancreatic islets and showed a similar level of accumulation. These results indicate that the content percentage of a polyethylene glycol-modified lipid in the lipid nanoparticles according to the present invention is preferably 1 mol % or more, more preferably 2 to 6 mol %, and even more preferably 2.5 to 5 mol %.

### Example 10

### 10. Investigation on Protective Effects of Nobiletin-Containing Lipid Nanoparticles on Pancreatic β Cells.

In this Example, lipid nanoparticles containing nobiletin (NOB), a methylated flavonoid contained in citrus fruit skin, were prepared as in Example 7 described above, and in vitro studies were conducted to determine whether the NOB-containing lipid nanoparticles were able to keep the protective effects of nobiletin on pancreatic β cells.

### [Preparation of Nobiletin (NOB)-Containing Lipid Nanoparticles]

Nobiletin powder (Ushio ChemiX Corporation) was dissolved in ethanol to prepare a stock solution. In this Example, a sample solution (final concentration: 11.5 mM) to be pumped into the microfluidic device was prepared in which DOPC was used as a phospholipid, cholesterol (Chol) was used as a sterol lipid, and DSG-PEG 2000 was used as a PEG-modified lipid with a composition of DOPC:Chol:DSG-PEG 2000:NOB=60:40:5:10 (by molar ratio). The total flow rate (TFR=150 mL/min) and flow rate ratio (FRR=1) of the sample solution and the water-soluble solution were adjusted, and NOB-containing lipid nanoparticles were prepared using similar materials and methods as in Example 7-1 using a nanoparticle manufacturing device. The total concentration of the components in the resulting dispersion containing the NOB-containing lipid nanoparticles was 5.75 mM. This was dialyzed against a PBS solution (pH 7.4) for 1 hour to obtain NOB-containing lipid nanoparticles in which the total concentration of the components was 4.32 mM. Table 12 below shows the average particle size, PDI, and the enclosure percentage of nobiletin of the resulting NOB-containing lipid nanoparticles. It is noted that the enclosure percentage of nobiletin was measured by a similar method as in Example 7.

TABLE 12

| Lipid | | | | Average | | Enclosure |
|---|---|---|---|---|---|---|
| Phospholipid | Sterol lipid | PEG-modified lipid | | particle | | percentage |
| (molar ratio) | (molar ratio) | (molar ratio) | Drug | size (nm) | PDI | (%) |
| DOPC | Cholesterol | DSG-PEG2000 | Nobiletin | 159 | 0.334 | 56.5 |
| (60 mol) | (40 mol) | (5 mol) | (10 mol) | | | |

The final concentration of nobiletin in the nobiletin-containing lipid nanoparticles was determined as 4.32 mM (the total concentration of the components)×8.7% (mol % of NOB in the components)×56.5% (the enclosure percentage of NOB)=0.212 mM.

### [Measurement of Antiapoptotic Effects of NOB Alone and NOB-Containing LNPs on Pancreatic β Cells]

Insulinoma INS-1 cells derived from rat pancreatic β cells were seeded into a 6-well dish at 8×10⁵ cells/well. This was cultured for 2 days in RPMI-1640 complete medium containing 11.1 mM glucose and 10% FBS. Two days after seeding, thapsigargin, an apoptosis inducer by inducing endoplasmic reticulum stress, was added to each well at a concentration of 300 nM, and nobiletin alone or NOB-containing LNPs prepared in this Example were added to each well for treatment at a nobiletin concentration of 10 M or 20 μM. After 14 hours of treatment, the cells were collected and subjected to SDS-PAGE, and protein expression in pancreatic β cells was analyzed by western blotting using a cleaved caspase-3 antibody as an indicator of apoptosis. Correction was made using the expression level of 3-actin as an internal standard.

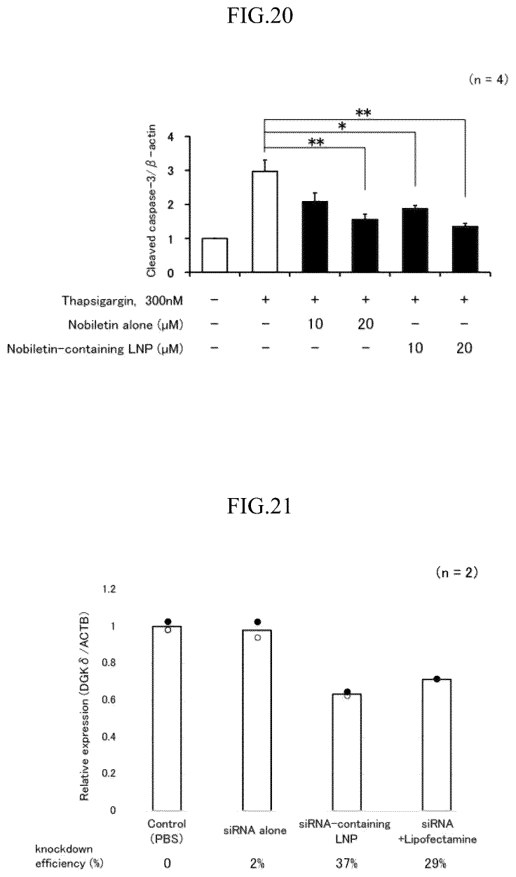

The results are shown in FIG. 20. Increase in the activity of the cleaved caspase 3 due to 14-hour treatment with thapsigargin, an apoptosis inducer by inducing endoplasmic reticulum stress was significantly suppressed by treatment with nobiletin-containing lipid nanoparticles, indicating that the nobiletin-containing lipid nanoparticles had antiapoptotic activities for pancreatic β cells. The effects were comparative with nobiletin alone. This reveals that the protective effects of nobiletin on pancreatic β cells are maintained even in the nobiletin-containing lipid nanoparticles similarly as nobiletin alone.

### Example 11

### 11. Investigation on Knockdown Effects of Nucleic Acid-Containing Lipid Nanoparticles in Pancreatic β Cells

In this Example, nucleic acid-containing lipid nanoparticles containing siRNA were prepared as in Example 8 described above, and in vitro studies were conducted to determine whether these siRNA-containing lipid nanoparticles were effective in suppressing gene expression of a target gene in pancreatic β cells. When diacylglycerol kinase δ (DGK δ), which is localized in the nuclei of pancreatic β cells, is suppressed, the proliferation of pancreatic β cells is promoted to ameliorate pathological conditions of diabetes. In this Example, to investigate the knockdown effects of the DGK δ gene in pancreatic β cells, DGK δ-targeting siRNA (antisense strand, 5′-UUU AGU AAG AUC CAG CAU CAC AUU C-3′ (SEQ NO: 1), Japan Bio Services Co., LTD.) was used as siRNA to prepare lipid nanoparticles containing DGK δ-targeting siRNA.

### [Preparation of Nucleic Acid-Containing Lipid Nanoparticles]

In this Example, DOPC was added as a phospholipid, cholesterol (Chol) was added as a sterol lipid, and DSG-PEG 2000 was added as a PEG-modified lipid, and D-Lin-MC3-DMA (MC3) was used as an ionized lipid. A sample solution (total lipid concentration: 11 mM) to be pumped to the microfluidic device was prepared with a composition of MC3:DOPC:Chol:DMG-PEG 2000=50:20:37:3 (by molar ratio). A solution consisting of 25 mM Acetate buffer (pH 4.0) and 450 mM NaCl was used a water-soluble solution, and DGK δ-targeting siRNA was mixed with that water-soluble solution (final concentration of DGK δ-targeting siRNA: 4.17 μM). It is noted that the N/P ratio was 6. The total flow rate (TFR=100 mL/min) and the flow rate ratio (FRR=3) of the sample solution and the water-soluble solution were adjusted, and lipid nanoparticles containing DGK δ-targeting siRNA were prepared using similar materials and methods as in Example 8-1 using a nanoparticle manufacturing device. The total concentration of lipids in the resulting dispersion containing the siRNA-containing lipid nanoparticles was 2.75 mM. This was dialyzed against MES (20 mM, pH 5.5) for 2 hours, and then dialyzed against a PBS solution (pH 7.4) overnight to adjust the final siRNA concentration in the lipid nanoparticles containing DGK δ-targeting siRNA to 3 μM. Table 13 below shows the average particle size, PDI, and the enclosure percentage of siRNA of the resulting lipid nanoparticles.

TABLE 13

| Lipid | | | | Average | | Enclosure |
|---|---|---|---|---|---|---|
| Ionized Lipid | Phospholipid | Sterol lipid | PEG-modified lipid | particle | | percentage |
| (molar ratio) | (molar ratio) | (molar ratio) | (molar ratio) | size (nm) | PDI | (%) |
| MC3 | DOPC | Cholesterol | DSG-PEG2000 | 148 | 0.255 | 97.0 |
| (50 mol) | (20 mol) | (37 mol) | (3 mol) | | | |

### [Knockdown of DGK δ Gene in Pancreatic β Cells]

MIN 6B cells derived from mouse pancreatic 3 cells were seeded into a 6-well dish at 8×10⁵ cells/well. This was cultured for 24 hours in D-MEM medium containing high glucose and 15% FBS. After 24 hours, the D-MEM medium was replaced with transformation medium (Opti-MEM®, from Thermo Fisher Scientific, Inc.), and the treatments were performed as follows: (1) the DGK δ-targeting siRNA only (naked siRNA), (2) the LNPs containing DGK δ-targeting siRNA prepared in this Example, and (3) a combination of the DGK δ-targeting siRNA and a transfection reagent (Lipofectamine® 2000, from Thermo Fisher Scientific, Inc.) were each added to each well for treatment to give an siRNA concentration of 150 nM. It is noted that PBS was added as a solvent control. The medium was replaced back with the D-MEM medium containing high glucose and 15% FBS 6 hours after the transfection treatments, and then cultured. Subsequently the cells were collected 48 hours after the transfection treatment, and total RNA was obtained using a total RNA preparation kit (NucleoSpin® RNA, Takara Bio Inc.). RT-qPCR was performed on the resulting total RNA to analyze whether expression of the DGK δ gene was suppressed.

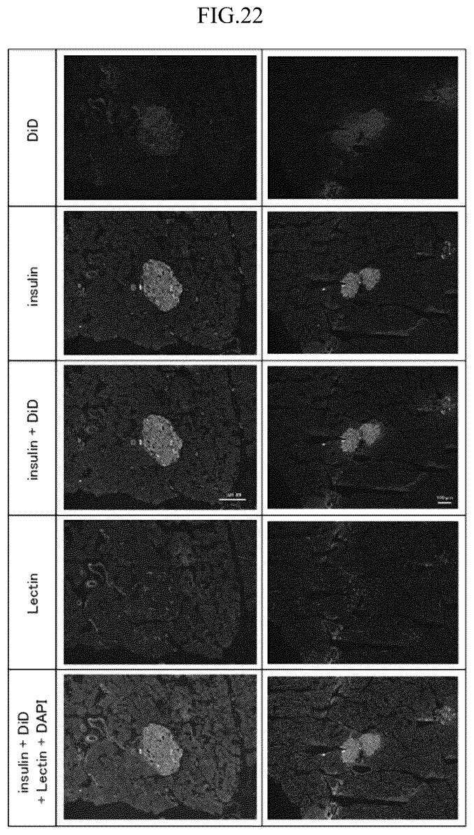

The results are shown in FIG. 21. These results indicate that the siRNA alone had little effect on suppression of the expression of the DGK δ gene as a target gene, but the siRNA-containing lipid nanoparticles suppressed the expression of the target gene at a higher rate than the test group in which a transfection reagent (Lipofectamine 2000) was used as a positive control. These reveal that the siRNA-containing lipid nanoparticles have sufficient knockdown effects in pancreatic β cells.

### Example 12

### 12. Distribution of Lipid Nanoparticles in Pancreatic 3 Cells

In this Example, whether the lipid nanoparticles according to the invention were reliably distributed in pancreatic β cells was investigated by performing immunohistochemistry of pancreatic β cells as insulin-positive cells using an anti-insulin antibody. In this Example, the lipid nanoparticles according to Example 12 were prepared using similar materials and methods as in Example 1 described above. Table 5 below shows the average particle size and PDI of the resulting lipid nanoparticles.

TABLE 14

| | | | PEG-modified | Average | |
|---|---|---|---|---|---|
| | Phospholipid | Sterol lipid | lipid | particle | |
| | (molar ratio) | (molar ratio) | (molar ratio) | size | PDI |
| Example 12 | DOPC | Cholesterol | DSG-PEG2000 | 152 nm | 0.262 |
| | (60 mol) | (40 mol) | (5 mol) | | |

For administration of the lipid nanoparticles, 11-week old male C57BL/6J mice (Japan SLC, Inc.) were used. These C57BL/6J mice were administered in tail vein with 200 μL (total amount of lipids: 5 μmol/mouse) of each of the DiD-labeled lipid nanoparticle dispersions prepared in this Example. DyLight 488-labeled lectin (Lycopersicon esculentum (Tomato) Lectin-LEA, DyLight 488 from Thermo Fisher Scientific, Inc.) as a vascular labeling reagent was diluted 4 fold with PBS, and 200 μL (50 μg in terms of LEL) was administered in tail vein 24 hours after administration. Mice were anesthetized by inhalation of isoflurane 5 minutes after administration of DyLight 488-labeled lectin, and blood was then drawn from inferior the vena cava, and the pancreas was isolated.

The isolated pancreas was immersed into and fixed with a solution of 4% paraformaldehyde and then impregnated with 10% sucrose for 4 hours, followed by 15% sucrose for 4 hours. It was then impregnated with 20% sucrose and allowed to stand overnight, and was dehydrated. Into a Cryomold, added and mixed were 300 μL of PBS and an O.C.T. Compound, to which the pancreas was then embedded. The specimen was then frozen in liquid nitrogen to obtain a frozen pancreatic specimen. The frozen pancreatic specimen was thinly sliced in a cryostat at a thickness of 5 μm, which was then attached to a poly-L-lysine-coated glass slide, and allowed to be air-dried under a light-shielded environment for 30 minutes. These were immersed in PBS for 10 minutes and then washed, which was repeated three times, and then blocking treatment was performed by immersion in a 3% BSA solution for 15 minutes. After blocking treatment, immersion in PBS for 10 minutes and washing was repeated three times. Next, a 2500-fold diluted anti-human insulin antibody (guinea pig) was used as a primary antibody for overnight treatment, and the next day, immersion in PBS for 10 minutes and washing was repeated three times. Subsequently, a 200-fold diluted goat anti-guinea pig immunoglobulin G (Alexa Fluor 555) was used as a secondary antibody for one-hour treatment. This was sealed with a nuclear staining sealant (DAPI-Fluoromount-G from Cosmo Bio Co., Ltd.). The slide was observed and analyzed with a fluorescence microscope (BZ-X810 from Keyence Corporation).

The results are shown by photographs in FIG. 22. “DiD” indicates DiD-labeled lipid nanoparticles which were stained red, and “insulin” indicates insulin-positive cells, i.e., pancreatic β αcells, which were stained white by anti-insulin antibody. It is noted that “lectin” indicates vascular tissues which were stained green, and “DAPI” indicates nuclei which were stained blue. These results demonstrate that a large number of DiD-labeled lipid nanoparticles showing red fluorescence were distributed in the same region as the insulin-positive cells, i.e., pancreatic β cells, which were stained white by the anti-insulin antibody. This indicates that the lipid nanoparticles according to the present invention reliably migrated and accumulated to pancreatic β cells upon intravenous administration.

The present invention is not limited to the above embodiments or Examples, but includes various alternatively designed and modified forms within its technical scope without departing from the spirit of the invention as defined in Claims.

### INDUSTRIAL APPLICABILITY

The present invention provides lipid nanoparticles that can efficiently deliver a drug to the pancreas. The lipid nanoparticles according to the present invention are widely useful in the pharmaceutical and medical fields and other industries because they can be used as, for example, medicaments for treating pancreatic diseases such as diabetes and pancreatic cancer.

1. Lipid nanoparticles used for delivering a drug to a target tissue, comprising:
(a) a phosphatidylcholine-type phospholipid having an unsaturated fatty acid chain with 16 to 24 carbon atoms, and
(b) a polyethylene glycol-modified lipid, and wherein
the target tissue is pancreas.

2. The lipid nanoparticles according to claim 1, wherein (a) the phospholipid is 1,2-dioleoyl-sn-glycero-3-phosphocholine (DOPC).

3. The lipid nanoparticles according to claim 1, wherein
the average particle size of the lipid nanoparticles is 100 nm to 200 nm,
the target tissue is a pancreatic islet of pancreas.

4. The lipid nanoparticles according to claim 1, wherein
the average particle size of the lipid nanoparticles is 10 nm to 50 nm,
the target tissue is an exocrine tissue of pancreas.

5. The lipid nanoparticles according to claim 1, further comprising (c) a sterol lipid.

6. The lipid nanoparticles according to claim 5, wherein
relative to the total amount of lipids constituting the lipid nanoparticles,
the content percentage of (a) the phospholipid is 40 mol % to 75 mol %,
the content percentage of (b) the polyethylene glycol-modified lipid is 0.01 mol % to 10 mol %,
the content percentage of (c) the sterol lipid is 25 mol % to 60 mol %.

7. The lipid nanoparticles according to claim 1, wherein
(b) the polyethylene glycol-modified lipid is distearoyl-rac-glycerol-PEG (DSG-PEG), 1,2-distearoyl-sn-glycero-3-phosphoethanolamine-N-[amino(polyethylene glycol)](DSPE-PEG), or 1,2-dimyristoyl-rac-glycero-3-methoxypolyethylene glycol (DMG-PEG).

8. The lipid nanoparticles according to claim 5, wherein
(c) the sterol lipid is cholesterol or a cholesterol derivative.

9. The lipid nanoparticles according to claim 5, further comprising
(d) an ionized lipid.

10. The lipid nanoparticles according to claim 1, containing
a drug for treating a pancreatic disease.

11. A medicament having the lipid nanoparticles according to claim 10 as an active ingredient.

12. A method of delivering a therapeutic agent to a target tissue of a patient, comprising administering to a patient a lipid nanoparticle comprising a therapeutic agent, the lipid nanoparticle, comprising:
(a) a phosphatidylcholine-type phospholipid having an unsaturated fatty acid chain with 16 to 24 carbon atoms, and
(b) a polyethylene glycol-modified lipid, and wherein
the target tissue is pancreas.

13. The method of claim 12, wherein (a) the phospholipid is 1,2-dioleoyl-sn-glycero-3-phosphocholine (DOPC).

14. The method of claim 12, wherein
the average particle size of the lipid nanoparticle is 100 nm to 200 nm,
the target tissue is a pancreatic islet of pancreas.

15. The method of claim 12, wherein
the average particle size of the lipid nanoparticle is 10 nm to 50 nm,
the target tissue is an exocrine tissue of pancreas.

16. The method of claim 12, wherein
the lipid nanoparticle, further comprising (c) a sterol lipid.

17. The method of claim 16, wherein
relative to the total amount of lipid constituting the lipid nanoparticle,
the content percentage of (a) the phospholipid is 40 mol % to 75 mol %,
the content percentage of (b) the polyethylene glycol-modified lipid is 0.01 mol % to 10 mol %,
the content percentage of (c) the sterol lipid is 25 mol % to 60 mol %.

18. The method of claim 12, wherein
(b) the polyethylene glycol-modified lipid is distearoyl-rac-glycerol-PEG (DSG-PEG), 1,2-distearoyl-sn-glycero-3-phosphoethanolamine-N-[amino(polyethylene glycol)](DSPE-PEG), or 1,2-dimyristoyl-rac-glycero-3-methoxypolyethylene glycol (DMG-PEG).

19. The method of claim 16, wherein
(c) the sterol lipid is cholesterol or a cholesterol derivative.

20. The method of claim 16, wherein
the lipid nanoparticle, further comprising
(d) an ionized lipid.
